(19) United States
(12) Patent Application Publication
(10) Pub. No.: US 2025/0025677 A1
(43) Pub. Date: Jan. 23, 2025

(54) INLET VALVES FOR A CARDIAC ASSIST DEVICE AND RELATED TECHNOLOGY

(71) Applicant: CardiacBooster B.V., Nijmegen (NL)

(72) Inventors: Daniël Immanuel Michaël van Dort, Nijmegen (NL); Florian Niklas Ludwig, Hilversum (NL); René Cornelis Henricus van der Burgt, Nijmegen (NL); Ferry van der Linde, Schijndel (NL); Jasper Gerhard Toebes, Nijmegen (NL); Patrick Griffin, Galway (IE); Tamas Jager, Galway (IE); David O'Reilly, Galway (IE); Shane Mulderrig, Galway (IE); Benedetta Sabiu, Arnhem (NL); Anisia Lauditi, Nijmegen (NL)

(21) Appl. No.: 18/906,419

(22) Filed: Oct. 4, 2024

Related U.S. Application Data
(63) Continuation of application No. PCT/EP2023/059293, filed on Apr. 6, 2023.
(60) Provisional application No. 63/328,295, filed on Apr. 7, 2022.

(51) Int. Cl. A61M 60/139; A61M 60/295; A61M 60/497

(52) U.S. Cl. CPC A61M 60/139; A61M 60/295; A61M 60/497

(57) ABSTRACT

A cardiac assist device includes a cup having a cup wall defining an inner cup volume, and an outflow element having an aperture for expelling a fluid during operation. A balloon has a balloon wall positioned inside the cup. A lumen is present for inflating and deflating the balloon during operation, creating a pumping operational mode and a filling operational mode, respectively. One or more one-way valves are arranged in the cup wall to allow the fluid to flow into the cup during the filling operational mode. One or more of the one-way valves comprise apertures and flaps arranged to close off the apertures during the pumping operational mode.

### CROSS-REFERENCE TO RELATED APPLICATION(S)

The present application is a continuation of International Application No. PCT/EP2023/059293, filed Apr. 6, 2023, which claims the benefit of priority to U.S. Provisional Application No. 63/328,295, filed Apr. 7, 2022, each of which is incorporated by reference herein in its entirety.

### TECHNICAL FIELD

The present technology is related to cardiac assist devices, such as mechanical devices implanted in the heart to improve blood flow.

### BACKGROUND

For some patients such as those suffering from cardiogenic shock or those undergoing high-risk percutaneous coronary interventions (PCI), a patient's heart function may be compromised such that the use of circulatory assist devices may be required to maintain adequate blood flows through the circulatory system.

Common types of circulatory assist devices include intra-aortic balloon pumps (IABP), extra-corporeal membrane oxygenation (ECMO) systems, and impeller-based blood pumps. IABPs are catheters having an inflatable balloon that can be placed in the descending aorta and cyclically inflated to displace the blood. ECMO systems include a venous catheter for removing deoxygenated blood from the venous system, an extracorporeal oxygenator and pump, and an arterial catheter for returning the blood to the arterial system, thus bypassing the heart. Impeller pump systems have a rotary impeller that can be placed in a chamber of the heart or in a major vessel and rotated at relatively high speed to propel blood through the circulatory system.

However, while offering some benefit in increasing blood flow and reducing load on the heart, currently available circulatory assist devices suffer from certain drawbacks. For example, IABPs may not improve flows adequately to support the patient when the heart is significantly compromised, such as during cardiogenic shock. As another example, ECMO systems may have higher morbidity associated with multiple catheterizations including bleeding, thrombus, and infection, as well as problems associated with membrane oxygenation including cognitive deficit and stroke. In addition, ECMO systems increase afterload, which is generally regarded as counterproductive. Impeller pump systems, if operated at higher speeds in order to produce higher flows as needed for certain patients, can result in excessive hemolysis. Furthermore, if impeller pumps are made larger to produce higher flows, the profile of such devices can be undesirably large, thereby inhibiting percutaneous delivery, increasing the risk of injury to cardiovascular structures and/or causing limb ischemia. As a result, current impeller-type pumps capable of providing high flows (which may be necessary for certain patients such as those in cardiogenic shock) are often too large for endovascular delivery, thus requiring surgical placement and risking undesirable levels of hemolysis.

What are needed, therefore, are improved cardiac assist devices.

### SUMMARY

Improved cardiac assist devices are disclosed. A cardiac assist device in accordance with at least some embodiments of the present technology comprises a cup having a cup wall comprising a first material and defining an inner cup volume, an outflow element connected with the cup wall and having an aperture in fluid communication with the inner cup volume for expelling a fluid during operation, and a balloon having a balloon wall comprising a second material and defining an inner balloon volume, the balloon being positioned inside the cup element free from the outflow element. A lumen (e.g., a lumen in a tube or a lumen within the cup wall) is present in fluid communication with the balloon for inflating and deflating the balloon during operation, creating a pumping operational mode and a filling operational mode, respectively. One or more one-way valves are arranged in the cup wall to allow the fluid to flow into the cup during the filling operational mode, wherein at least some of the one or more one-way valves include a portion of the cup wall that defines one or more apertures, and a flap arranged to close off the one or more apertures during the pumping operational mode.

Cardiac assist devices in accordance with at least some embodiments of the present technology allow for efficient operation, have high pumping capacity, have fast opening and closing times of the inlet valves, have low resistance during inflow of fluid, have low leakage during filling, have low leakage during pumping, and/or have one or more other advantages relative to the prior art.

### BRIEF DESCRIPTION OF THE DRAWINGS

Many aspects of the present technology can be better understood with reference to the following drawings. The relative dimensions in the drawings may be to scale with respect to some embodiments of the present technology. With respect to other embodiments, the drawings may not be to scale. The drawings may also be enlarged arbitrarily. For clarity of illustration, reference-number labels for analogous components or features may be omitted when the appropriate reference-number labels for such analogous components or features are clear in the context of the specification and all of the drawings considered together. Furthermore, the same reference numbers may be used to identify analogous components or features in multiple described embodiments.

FIGS. 1A and 1B are side views of an example cardiac assist device in accordance with at least some embodiments of the present technology.

FIG. 1C is a schematic of an example cardiac assist device placed in a heart of a patient, in accordance with at least some embodiments of the present technology.

FIGS. 2A-2H are schematic cross-sectional side views of various example one-way inlet valves of a cardiac assist device in accordance with at least some embodiments of the present technology.

FIG. 3 is a top plan view of an example inlet valve of a cardiac assist device in accordance with at least some embodiments of the present technology.

FIG. 4A is a top plan view of an example inlet valve of a cardiac assist device in accordance with at least some embodiments of the present technology. FIG. 4B is a schematic side view of an example cardiac assist device including an inlet valve arrangement including the inlet valve depicted in FIG. 4A.

FIG. 5 is a side view of an example cardiac assist device in accordance with at least some embodiments of the present technology.

FIG. 6 is a side view of an example cardiac assist device in accordance with at least some embodiments of the present technology.

FIG. 7 is an exploded perspective view of an example arrangement of one-way inlet valves of a cardiac assist device in accordance with at least some embodiments of the present technology.

FIGS. 7A-7C are top plan views of different example arrangements of one-way inlet valves of cardiac assist devices in accordance with at least some embodiments of the present technology.

FIG. 8 is an exploded perspective view of an example arrangement of one-way inlet valves of a cardiac assist device in accordance with at least some embodiments of the present technology.

FIG. 9 is a top plan view of an example arrangement of one-way inlet valves of a cardiac assist device in accordance with at least some embodiments of the present technology.

FIG. 10A is a top plan view of an example arrangement of one-way inlet valves of a cardiac assist device in accordance with at least some embodiments of the present technology. FIGS. 10B and 10C are top plan views of components of the series of one-way inlet valves depicted in FIG. 10A. FIGS. 10D and 10E are side views of an inlet valve in the example arrangement depicted in FIG. 10A, in closed and open states, respectively.

FIG. 11A is a top plan view of an example arrangement of at least one inlet valve of a cardiac assist device in accordance with at least some embodiments of the present technology. FIGS. 11B and 11C are side views of an inlet valve in the example arrangement depicted in FIG. 11A, in closed and open states, respectively.

FIG. 12A is a top plan view of an example inlet valve with reinforcement features of a cardiac assist device in accordance with at least some embodiments of the present technology. FIGS. 12B-12E are side views of example reinforcement features for an inlet valve in a cardiac assist device.

FIG. 13 is a top plan view of an example inlet valve with reinforcement features of a cardiac assist device in accordance with at least some embodiments of the present technology.

FIGS. 14A-14E are top plan views of apertures of example one-way inlet valves of a cardiac assist device in accordance with at least some embodiments of the present technology.

FIG. 15 is a schematic perspective view of portions of an example one-way inlet valve of a cardiac assist device in accordance with at least some embodiments of the present technology.

FIGS. 16-18 are top plan views of example one-way inlet valves of a cardiac assist device in accordance with at least some embodiments of the present technology.

FIGS. 19A-19C are top plan views of example one-way inlet valves of a cardiac assist device in accordance with at least some embodiments of the present technology.

FIGS. 20A-20E are side views of example one-way inlet valves of a cardiac assist device in accordance with at least some embodiments of the present technology.

FIG. 21 is a top plan view of an example arrangement of one-way inlet valves of a cardiac assist device in accordance with at least some embodiments of the present technology.

### DETAILED DESCRIPTION

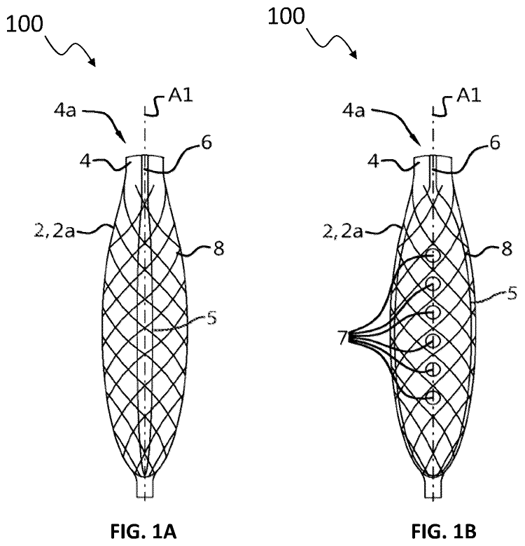

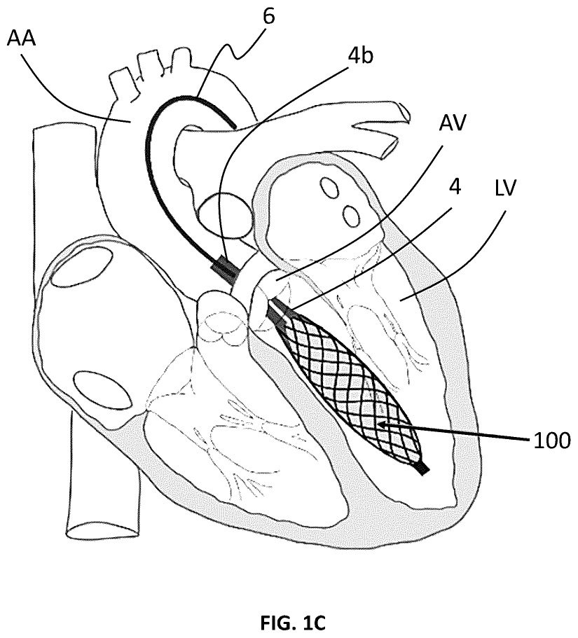

Specific details of several embodiments of the present technology are disclosed herein with reference to FIGS. 1-11. It should be noted, in general, that other embodiments in addition to those disclosed herein are within the scope of the present technology. For example, embodiments of the present technology can have different configurations, components, and/or operations than those disclosed herein. Moreover, a person of ordinary skill in the art will understand that embodiments of the present technology can have configurations, components, and/or operations in addition to those disclosed herein and that these and other embodiments can be without configurations, components, and/or operations disclosed herein without deviating from the present technology.

Intra-lumen cardiac assist devices (also referred to herein as an intravascular blood pump) are disclosed. These devices can provide circulatory assistance to a patient by pumping blood from a cardiovascular lumen (e.g. the left ventricular chamber) with a sufficiently high flowrate and efficient placement. Cardiac assist devices in accordance with at least some embodiments of the present technology allow an efficient inflow of fluid (e.g., blood) as well as proper sealing during a pumping phase. Among other features, inlet valves described herein may provide proper one-way valve functionality, allow proper timing of filling and pumping stages, provide a low inflow resistance during filling, and/or provide a low leakage rate during pumping.

FIGS. 1A and 1B are side views of a cardiac assist device 100 according to at least some embodiments of the present technology. As shown in FIG. 1C, the cardiac assist device 100 can be placed, for example, in the left ventricle LV of a patient. The device comprises a cup 2 (alternatively referenced herein as a shell) having a cup wall 2a defining an inner cup volume. In the embodiment shown in FIGS. 1A and 1B, the cup wall 2a is supported by a skeleton structure 8. The skeleton structure 8 can provide structural rigidity to the cardiac assist device while also allowing the cardiac assist device to take a very narrow shape, elongated along a longitudinal axis A1, that facilitates intravascular delivery to a body lumen (e.g., the left ventricle of the heart) using a catheter or the like. For example, the cup can be configured to move intravascularly along a delivery path parallel to the longitudinal axis A1. In at least some embodiments, the skeleton structure 8 includes struts arranged in a mesh pattern that defines interstices (alternatively referenced herein as openings). The mesh pattern can define any regular or irregular pattern of interstices that can be collapsed or expanded to move the skeleton structure 8 between a low-profile delivery state and an expanded pumping state. The individual interstices can be polygonal, rounded, or have another suitable form. In at least some cases, the interstices are diamond shaped. Additional details and examples of the cup 2 can be found, for example, in PCT Publication No. WO2022/235152, which is incorporated herein in its entirety by reference.

An outflow element 4 is connected with the cup wall 2a and has an outlet aperture 4a in fluid communication with the inner cup volume for expelling a fluid from the cup 2 during operation of the device. For example, as shown in FIG. 1C, when the device 100 is placed in a left ventricle LV of a patient's heart, the cup 2 can be positioned such that the outflow element 4 is configured to expel fluid from the cup 2 and out of the heart. The outflow element 4 can include an outflow nozzle 4b near or at the outlet aperture. The outflow nozzle 4b can, for example, be configured to extend from the cup 2 and pass across the aortic valve into the ascending aorta. In some embodiments, the outflow nozzle 4b can be configured to collapse and seal the outlet aperture 4a when pressure within the outflow nozzle 4b is exceeded by the pressure outside the outflow nozzle 4b. Additionally or alternatively, the outflow nozzle 4b or other portion of the outflow element 4 can include an outlet valve (e.g., a one-way valve). Additional details and examples of the outflow element 4 can be found, for example, in PCT Publication No. WO2022/235152, which was incorporated above.

The device can further include at least one volume displacement member such as a balloon 5 having a balloon wall comprising a second material and defining an inner balloon volume. The balloon 5 can be at least partially contained in the cup 2 (e.g., in the inner cup volume). In at least some cases, the balloon 5 is positioned inside the cup free from the outflow element 4. FIG. 1A shows the cardiac assist device with the balloon 5 deflated, and FIG. 1B shows the cardiac assist device with the balloon 5 inflated. Thus, the balloon 5 can be configured to move between a first state and a second state, with the balloon 5 being more inflated in the second state than in the first state. The device can include a tube 6 in fluid communication with the balloon 5 for inflating and deflating the balloon 5 during operation of the cardiac assist device. For example, the tube 6 can be configured to inflate the inner balloon element 5 (toward the second state as shown in FIG. 1B) in a pumping operational mode of the device, and deflate the inner balloon element 5 (toward the first state as shown in FIG. 1A) in a filling operational mode of the device. Additionally or alternatively, any suitable structure (e.g., cup wall with a lumen in the cup wall) with a lumen can be in fluidic communication with the balloon for inflating and deflating the balloon element during operation of the cardiac assist device. In at least some embodiments, the balloon 5 is positioned inside the cup 2 free from the outflow element 4.

The cardiac assist device can include one or more inlet valves 7 to allow fluid (e.g., blood) to enter the cup (e.g., the inner cup volume) during the filling operational mode of the device. At least some of the inlet valves can be a one-way valve, so as to substantially prevent fluid inside the cup from exiting the cup element via the one-way inlet valve(s) during the pumping mode of the device.

In general, when the cardiac assist device 100 is placed in a patient's heart (e.g., the left ventricle), the cup 2 and balloon 5 can cooperate to alternately fill the cup 2 with fluid via the one or more inlet valves 7 in the filling operational mode, and expel the fluid from the cup 2 via the outflow element 4 in the pumping operational mode. Specifically, deflation of the balloon in the filling operational mode of the device 100 urges the outflow nozzle 4b (and/or an outlet valve in the outflow element 4) to close and the inlet valve(s) 7 to open, thereby enabling fluid to enter and fill the cup 2 via the inlet valve(s) 7 (e.g., drawing blood from the LV through the inlet valve(s) 7 into the cup 2). Furthermore, inflation of the balloon in the pumping operational mode of the device 100 urges the outflow nozzle 4b to open and the inlet valve(s) 7 to close, thereby enabling fluid to exit the cup 2 through the outflow element 4 (e.g., expelling blood from the cup 2 through the outflow nozzle 4b and outflow aperture 4a). In some embodiments, as further described herein, the inlet valve(s) 7 can include one or more features (e.g., reinforcement(s), anchor element(s), etc.) for reducing the closure response time of the inlet valve(s) 7 and improving the efficiency of the cardiac assist device in the pumping operational mode. The pumping actuation frequency for inflating and deflating the balloon can be a high actuation frequency that can be dependent in part on the internal fluid volume that can be contained in the cup 2 outside of the balloon 5, and/or total desired cardiac flow. In some example embodiments, the pumping actuation frequency and/or frequency of inlet valve(s) 7 closing can be at least about 100 beats per minute, or at least about 300 beats per minute, such as in combination with an internal fluid volume (also referred to herein as stroke volume) of between about 0.3 ml and about 5 ml for each pump cycle. For example, in some embodiments, some or all of the inlet valve(s) 7 in an example cardiac assist device can be configured to transition from an open state to a closed state at least about 100 times per minute (i.e., at least about 1.66 times per second), and some or all of the inlet valve(s) 7 can be configured to transition from an open state to a closed state in about 0.6 seconds or less. As another example, in some embodiments, some or all of the inlet valve(s) 7 in an example cardiac assist device can be configured to transition from an open state to a closed state at least about 300 times per minute (i.e., at least about 5 times per second), and some or all of the inlet valve(s) 7 can be configured to transition from an open state to a closed state in about 0.2 seconds or less.

Various examples of inlet valves and inlet valve configurations are described herein. For example, in some embodiments, a one-way inlet valve can include at least one inlet valve aperture and at least one flap. In some embodiments, the inlet valve apertures can be circular, elliptical, diamond-shaped, or slit-shaped, for example. In some example embodiments, each of the inlet valve apertures can have a size (e.g., diameter or width) ranging between about 0.2 mm and about 3 mm. Each of the inlet valve apertures can have the same size and/or shape, or at least some of the inlet valve apertures can have different sizes and/or shapes.

The flap functions to limit fluid flow through the inlet valves via the aperture. In some embodiments, the flap can, in general, include a leaflet or other aperture-covering body that has an attached portion that is attached to the cup wall, and a free portion that is movable relative to the attached portion in a hinging, pivoting, or swinging type of motion. For example, the flap can include a leaflet. During the filling operational mode of the device, the flap can be configured to expose the inlet valve aperture(s) or otherwise enable fluid passage through the inlet valve aperture(s) into the cup. In contrast, during the pumping operational mode of the device, the flap can be configured to cover the inlet valve aperture(s) or otherwise substantially prevent fluid passage through the inlet valve aperture(s) out of the cup. For example, a pressure differential across the cup wall caused by inflation of the balloon in the pumping operation mode can at least in part cause the flap to cover the inlet valve aperture(s). As another example, physical engagement between the inflating balloon and the flap (e.g., when the balloon wall contacts and urges the flap radially outward against the cup wall) can additionally or alternatively cause the flap to cover the inlet valve aperture(s).

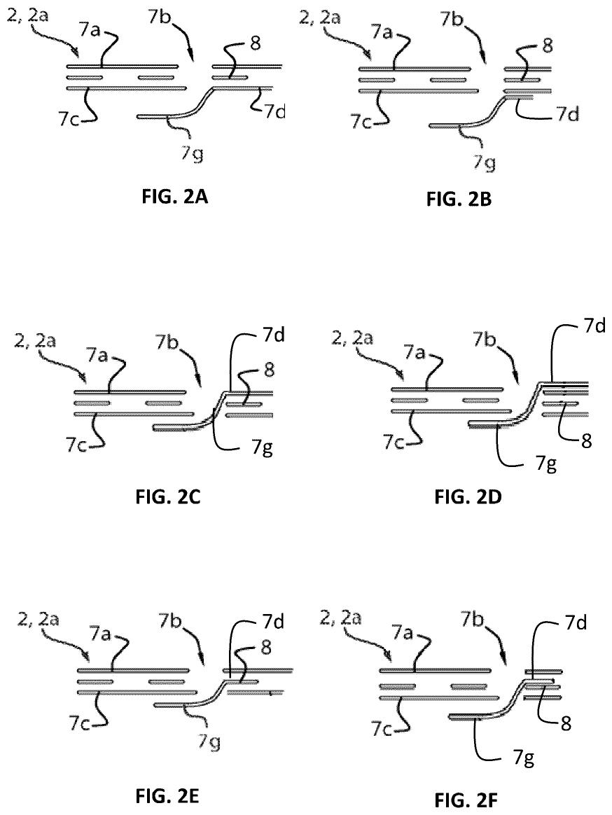

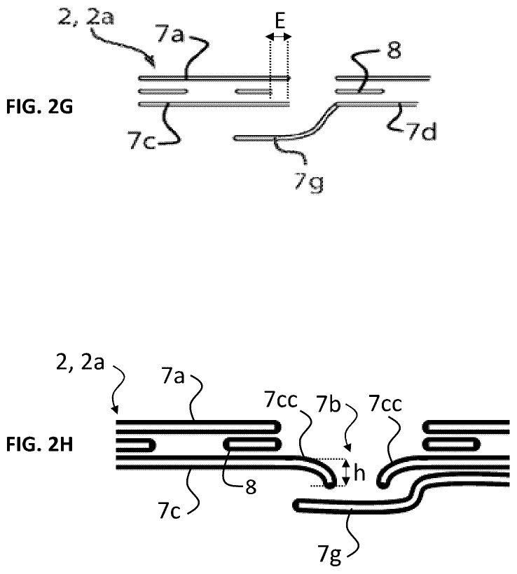

FIGS. 2A-2H are schematic cross-sectional side views of various example one-way inlet valves 7 of a cardiac assist device in accordance with at least some embodiments of the present technology. With reference to FIGS. 1A-2H together, the inlet valve 7 can comprise a portion of the cup wall 2a that defines one or more apertures 7b, and a flap 7g. The flap 7g can be arranged to permit fluid to flow into the cup 2 through the one or more apertures 7b during the filling operational mode of the device. Furthermore, the flap 7g can be arranged to close off the one or more apertures 7b during the pumping operational mode of the device. In the examples shown in FIGS. 2A-2H, the portion of the cup wall 2a at the inlet valve 7 includes an outer layer 7a and an inner layer 7c encasing the skeleton 8 therebetween. For example, the inner layer 7c and the outer layer 7a can be membranes carried by the struts of the skeleton structure 8. In other embodiments, the portion of the cup wall 2a at the inlet valve 7 can include no layers on either side of the skeleton 8, a single layer on one side of the skeleton 8 (e.g., only the inner layer 7a or only the outer layer 7c), or more than two layers on one or both sides of the skeleton 8. For example, the struts of the skeleton can form the entirety of the cup wall 2a. In these and other embodiments, the cup wall 2a can include integral webbing between the struts and the apertures 7b. For example, the cup wall 2a can include a molded polymeric structure including struts and apertures with webbing therebetween. Alternatively, the struts themselves can define the apertures 7b. For example, the interstices can be equivalent to the apertures 7b.

The flap 7g can be an integrally formed portion (e.g., a partial cutout) of the inner layer 7c (e.g., FIG. 2A), a separately formed structure attached to the inner layer 7c (e.g., FIG. 2B), an integrally formed portion (e.g., a partial cutout) of the outer layer 7a (e.g., FIG. 2C), a separately formed structure attached to the outer layer 7a (e.g., FIG. 2D), an integrally formed portion of the skeleton 8 (e.g., FIG. 2E), a separately formed structure attached to the skeleton 8 (e.g., FIG. 2F), or have another suitable form that allows the flap to move independently relative to the portion of the cup wall 2a at the one-way valve 7 to open and close the aperture 7b.

With reference again to FIGS. 1A-2H together, moving the balloon 5 from the second state (FIG. 1B) toward the first state (FIG. 1A) can tend to move fluid into the cup 2 through the inlet valve 7 via the aperture 7b, in a filling operational mode similar to that described above. In at least some embodiments, the aperture 7b is at a given one of the interstices defined by the struts. In these and other embodiments, the struts can provide structural support to a perimeter of the aperture 7b such that the aperture 7b remains relatively stationary during operation of the cardiac assist device. A distance between an inner edge of the struts at the given interstice and an outer edge of the aperture 7b (distance E shown in FIG. 2G) can be selected to enhance this structural support without unduly compromising a free-passage area through the one-way inlet valve 7. In at least some cases, this distance is within a range from 1 mm to 3 mm around at least 75% of a full perimeter of the aperture 7b. In addition or alternatively, this distance can be consistent around all or a portion of the aperture 7b. For example, this distance can vary less than 50% around at least 75% of a full perimeter of the aperture 7b. Thus, the shape of the aperture 7b can correspond to the shape of the given interstice.

In at least some embodiments, the selection of materials of the cup wall 2a and flap 7g can help enable the capacity of the cardiac assist device to withstand the pressures during operation (e.g., high-frequency operation). In addition or alternatively, inlet valves 7 in accordance with at least some embodiments of the present technology are configured to withstand a pressure gradient of at least 200 mmHg, or at least 300 mmHg (e.g., between about 300 mmHg and about 500 mmHg) in the cup, such as during the pumping operational mode, which will introduce positive pressure in the cup.

In at least some embodiments of the present technology, the material of the cup wall 2a (e.g., the outer layer 7a of the cup wall) and the material of the flap 7g are selected to provide case of manufacture and/or to provide sufficient strength to withstand forces during operation of the one-way valve 7. In some cases, the total open surface area of the apertures 7b (whether covered or uncovered by a flap 7g) in the one or more one-way valves 7 is at least 50 mm², e.g., at least 150 mm², or at least 300 mm². When present in a cardiac assist device with a pumping capacity (i.e. the difference between the inner volume of the cup wall 2a and the volume of the inner balloon element 5 when inflated and deflated) of, for example, between 1 ml and 12 ml per stroke, the total surface area of the apertures 7b can provide for a sufficiently high filling speed of the cardiac assist device to allow sustained operation. In some embodiments, one or more features of the inlet valves disclosed herein may cause the filling pressure of the cup 2 to be not lower than negative 200 mmHg. To prevent a pressure of no less than about negative 200 mmHg in the cup 2 during the filling operational mode, the inlet valve(s) 7 can be configured to provide an inflow of fluid into the cup that balances against (e.g., accommodates the volumetric debit of) balloon deflation inside the cup. For example, in some embodiments, the filling pressure of the cup is generally the result of how well (i) net balloon deflation capacity (which can be dependent upon, for example, inner balloon volume and balloon inflation/deflation cycling speed) and inflow capacity of the cup 2 (e.g., approximately inner cup volume not occupied by the deflated balloon), are balanced by (ii) an ideally low resistance of flow into through the one or more inlet valves (which can be dependent upon, for example, stiffness of the inlet valves 7, overall area of the apertures 7b, location of the inlet valves 7, and/or the nature of the connection 7 attaching the inlet valves 7 to the cup wall). For example, a total surface area of the apertures 7b of at least 120 mm² was found to provide a sufficient high filling speed during the filling operational mode of a cardiac assist device in accordance with at least some embodiments of the present technology. In some examples, a cardiac assist device can include a total surface area of the apertures 7b of at least 150 mm², at least 175 mm², at least 200 mm², or at least 300 mm². Furthermore, the total surface area of the apertures 7b in the one or more one-way valves 7 can be at least 10%, e.g. at least 25% of an outer surface area of the cup wall 2a. Having a sufficiently large amount of the outer surface of the device (e.g., the surface of the cup wall 2a) able to open and have an inflow of surrounding fluid during the filling operational mode can facilitate proper functioning of the cardiac assist device. The distribution of the apertures 7b over the cup wall 2a can, for example, be uniform. This can be useful, for example, to reduce or prevent areas of low flow and/or to maintain an inflow of fluid even in case of partial blockage.

In some example embodiments, a cardiac assist device can have a stroke volume of about 3 ml and a pumping actuation frequency of about 1500 beats per minute (25 Hz, with a period of about 40 ms), where inflow occurs over half of the period (relative inflow period of 50%). In these embodiments, the inlet valves 7 can be configured to provide an average inflow of about 3 ml over 20 ms into the cup, or a volumetric inflow rate (Q) of about 150 ml/s or more. Assuming a target pressure drop (P) across the cup wall of about 100 mmHg (that is, filling pressure of no less than about negative 100 mmHg, or about 13,300 Pa), the effective total valve resistance (TVR) is given by Equation 1 below:

$$TVR = \frac{P}{Q} = \frac{13 , 000 Pa}{150 ml / s} \qquad ( 1 )$$

In these examples, TVR is approximately 1×10⁸ kg/(m⁴ s). However, it should be understood that in other examples, the TVR may be lower or higher.

For example, in some embodiments, a cardiac assist device can have a stroke volume of about 3 ml and a pumping actuation frequency of about 300 beats per minute (5 Hz, with a period of about 200 ms), with a relative inflow period of 50%. In these embodiments, the inlet valves 7 can be configured to provide an average inflow of about 3 ml over 100 ms into the cup, or a volumetric inflow rate (Q) of about 30 ml/s or more. Assuming a target pressure drop (P) across the cup wall of about 100 mmHg, the effective TVR according to Equation 1 would be approximately 5×10⁸ kg/(m⁴s).

Furthermore, in some embodiments, a cardiac assist device can have a stroke volume of about 3 ml and a pumping actuation frequency of about 500 beats per minute (8.3 Hz, with a period of about 120 ms), with a relative inflow period of 50%. In these embodiments, the inlet valves 7 can be configured to provide an average inflow of about 3 ml over 60 ms into the cup, or a volumetric inflow rate (Q) of about 50 ml/s or more. Assuming a target pressure drop (P) across the cup wall of about 100 mmHg, the effective TVR according to Equation 1 would be approximately 3×10⁸ kg/(m⁴s).

Furthermore, in some embodiments, a cardiac assist device can have a stroke volume of about 3 ml and a pumping actuation frequency of about 10,000 beats more minute (166.67 Hz, with a period of about 6 ms), with a relative inflow period of 50%. In these embodiments, the inlet valves 7 can be configured to provide an average inflow of about 3 ml over 3 ms into the cup, or a volumetric inflow rate (Q) of about 1000 ml/s or more. Assuming a target pressure drop (P) across the cup wall of about 100 mmHg, the effective TVR according to Equation 1 would be approximately 0.15×10⁸ kg/(m⁴ s).

Accordingly, in some embodiments, TVR in a cardiac assist device having a pumping actuation frequency between about 300 beats per minute and about 10,000 beats per minute can range between about 5×10⁸ kg/(m⁴s) and about 0.15×10⁸ kg/(m⁴ s). In some embodiments, TVR in a cardiac assist device having a pumping actuation frequency between about 300 beats per minute and about 1500 beats per minute can range between about 5×10⁸ kg/(m⁴s) and about 1×10⁸ kg/(m⁴ s). In some embodiments, TVR in a cardiac assist device having a pumping actuation frequency between about 300 beats per minute and about 500 beats per minute can range between about 5×10⁸ kg/(m⁴ s) and about 3×10⁸ kg/(m⁴s).

In general, pressure drop across the inlet valve(s) depends on the total valve resistance (which is a function of at least the number of valves and their size and/or shape), and the stroke volume of the cup to be filled during device operation. For example, in some embodiments, to prevent negative filling pressure of no less than about negative 100 mmHg in a cardiac assist device with a stroke volume of about 3 ml having a balloon that deflates over a 10 ms period, the cardiac assist device has an overall aperture surface area of the inlet valves 7 of at least about 100 mm² providing sufficient inflow without excessive total valve resistance.

The flap 7g and cup wall 2a can comprise materials with different durometer values. For example, the flap 7g can comprise a material with a durometer value lower than a durometer value of the cup wall 2a. In these and other cases, an average stiffness of the cup wall 2a at a given coaptation area can be greater than an average stiffness of a portion of the flap 7g configured to contact the cup wall 2a at the coaptation area. In one example, the cup wall 2a is made at least primarily from a material having a durometer value of 72 D Shore, and the flap 7g is made at least primarily form a material having a durometer value of 85 A Shore. Materials with these characteristics may enhance scaling of the one-way valve 7 during the pumping operational mode of the cardiac assist device, such as by providing a sufficient stiffness of the material surrounding aperture 7b combined with sufficient flexibility of the material of the flap 7g to seal off the aperture 7b without the flap 7g bulging through the aperture 7b.

In some embodiments, the flap 7g and connection 7d can be arranged to impart a direction of flow of the fluid during the filling operational mode within the cup wall 2a. Furthermore, in embodiments in which multiple inlet valves 7 are present, the direction of flow from a given inlet valve 7 can influence the behavior of an adjacent inlet valve 7. This can enhance the closing speed of the inlet valves 7 during a change from a filling operational mode to a pumping operational mode. Furthermore, the closing speed of the inlet valves 7 can be relevant to achieving a low leakage rate. Leakage can occur when the inlet valve 7 is slow to close off and seal the aperture 7b during a transition from the filling operational mode to the pumping operational mode. Inlet valves 7 in accordance with at least some embodiments of the present technology allow a leakage rate of less than 0.5 ml per stroke, with a stroke being opening and closing of the inlet valve 7. This can be sufficiently low to cause a sustained outflow of fluid from the cardiac assist device. Examples of inlet valves imparting a direction of inflow and/or enhancing closing speed and/or valve scaling are described in further detail herein.

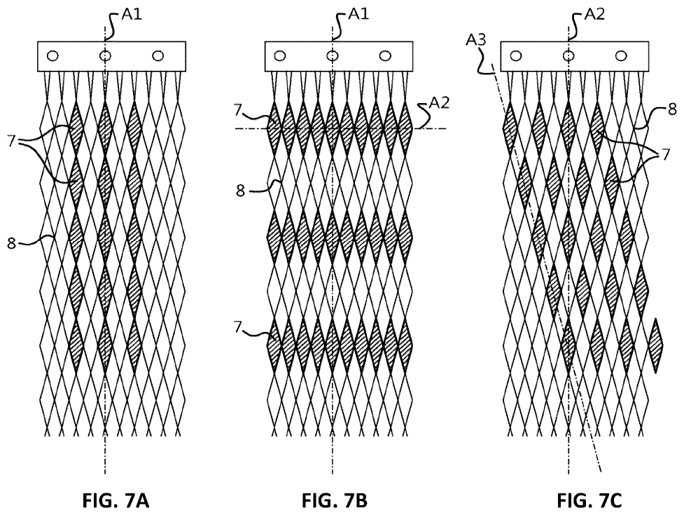

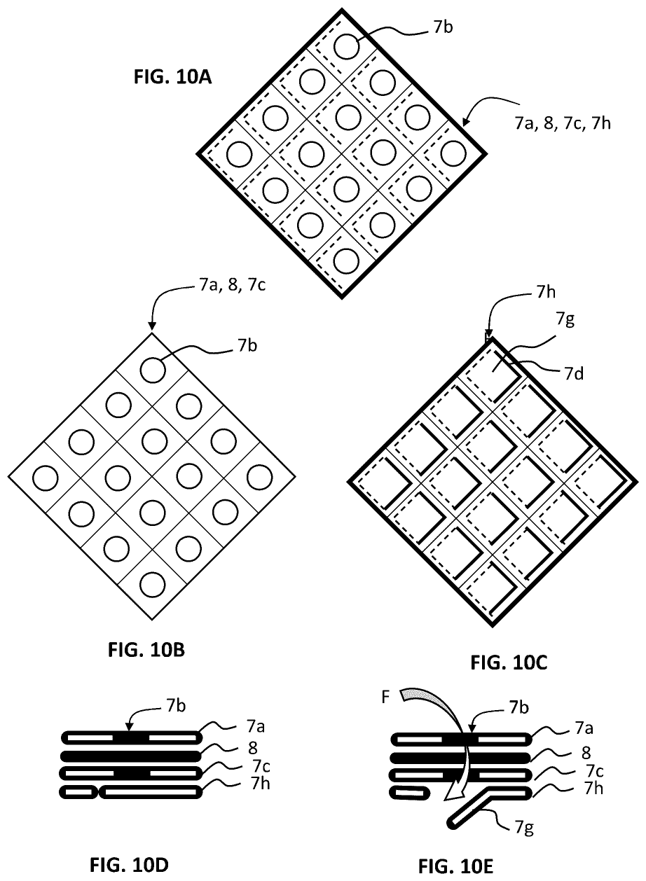

In some embodiments, a cardiac assist device can include an inlet valve arrangement having a flap-to-inlet valve aperture ratio of about 1:1 (e.g., each flap 7g covers and exposes a single respective aperture 7b). However, in some embodiments, an inlet valve arrangement can include at least one flap 7g that is configured to cover and expose multiple apertures 7b (e.g., two, three, four, or more), for example such as the inlet valve arrangements shown in and described herein with respect to FIGS. 7 and 10A-10E.

In some embodiments, at least a portion of the perimeter of the aperture 7b can be flared inwards, so as to help guide or otherwise facilitate a smoother flow pattern of fluid in an inflow direction into the cup 2 through the inlet valve, while discouraging or limiting fluid flow in an opposite, outflow direction out of the cup 2 through the inlet valve. For example, FIG. 2H illustrates an example inlet valve in which the inner layer 7c at the perimeter or edge of the aperture 7b at is flared inwards. However, it should be understood that other layers of the cup wall (e.g., skeleton 8, outer layer 7a) can additionally or alternatively include one or more inwardly flaring profiles at the perimeter or edge of the aperture 7b. In some example embodiments, as shown in FIG. 2H, the offset depth or height h of the flare can be between about 0.1 mm to about 2 mm, or about 0.5 mm.

In the cross-sectional views of FIGS. 2A-2H, the skeleton structure 8 is shown between the outer layer 7a and the inner layer 7c. The illustrated separation between the inner layer 7c, the outer layer 7a, and the skeleton structure 8 in FIGS. 2A-2H is for clarity of illustration. As discussed above, the inner layer 7c, the outer layer 7a, and the skeleton structure 8 can be integrated and/or otherwise at least partially connected to each other to form the cup wall 2a. The structure and/or composition of the skeleton structure 8 in combination with the outer layer 7a and/or the inner layer 7c can provide sufficient rigidity to support stable positioning of the flap 7g relative to the aperture 7b during operation of the inlet valve 7. This can be useful, for example, to reduce leakage through the aperture 7b when the inlet valve 7 is closed. In addition or alternatively, stable positioning of the flap 7g can promote more efficient and faster opening and closing of the inlet valve 7. In at least some embodiments, the outer layer 7a, the inner layer 7c, and/or the flap 7g comprise polymer materials (e.g., polymer fibers), carbon materials (e.g., nanocarbon fibers), and/or glass materials (e.g., glass fibers).

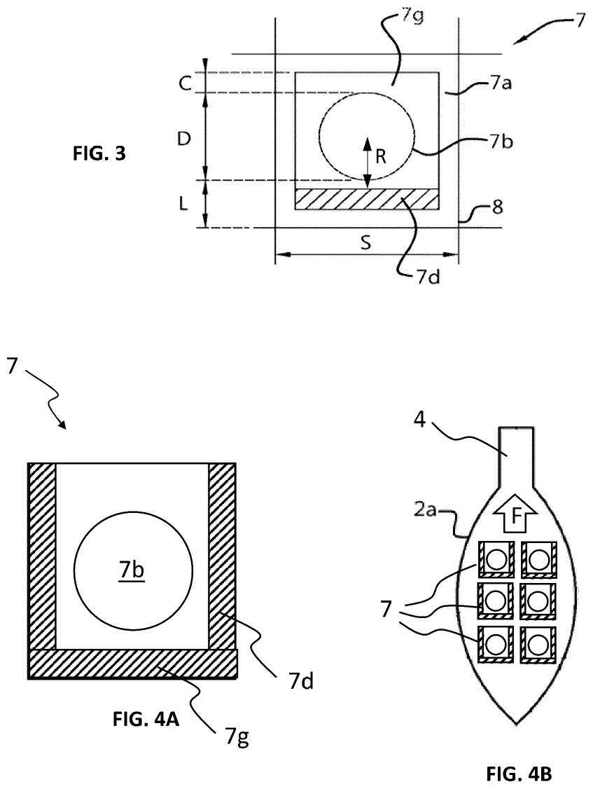

The flap 7g can be connected to (e.g., integrally joined with, or coupled to) the cup wall 2a at a connection 7d, which can also act as a living hinge. In some embodiments, the connection 7d is located adjacent to the aperture 7b at a hinge portion of the flap. The surface area of the connection 7d can have any suitable shape. For example, as shown in FIG. 3, the connection 7d may be rectangular (e.g., a rectangular strip extending along an attachment line). Additionally or alternatively, the connection 7d can include one or more points, lines, arcs, or other suitable shapes next to the aperture 7b and/or along at least a portion of the perimeter of the flap 7g.

Generally, in some embodiments, the connection 7d can extend along at least one attachment line whose orientation helps direct or steer fluid flow through the inlet valve 7 toward a particular direction. For example, direction of fluid flow through the inlet valve 7 is generally away from the attachment line(s). For an inlet valve having a single attachment line, the resulting direction of fluid flow through that inlet valve will be generally perpendicular and away from the single attachment line. For example, FIG. 3 illustrates an example inlet valve 7 having a connection 7d extending along an attachment line that is adjacent a lower side of the inlet valve aperture 7b and a lower side of the flap 7g, such that the flap 7g is configured to hinge around the attachment line and open inward relative to the page. When the flap 7g hinges inward in this manner, the inlet valve 7 is opened and enables flow through the aperture 7b primarily in a direction that is generally away from the attachment line (i.e., upwards, in the orientation shown in FIG. 3).

Although FIG. 3 illustrates an inlet valve 7 having a connection 7d extending along an attachment line on only one side of the flap 7g, it should be understood that the in some embodiments, the connection 7d can extend adjacent to any suitable portion of the perimeter of the flap 7g. Generally, the resulting direction or pattern of fluid flow through such inlet valves will be the direction that is a resultant of the individual vectors directed away from each respective attachment line. For example, FIGS. 4A and 4B illustrate an example arrangement of inlet valves 7 in a cardiac assist device, where an inlet valve 7 includes a flap 7g that is connected to the cup wall 2a via an upside-down “U”-shaped connection 7d extending around three sides (bottom and two lateral sides) of the aperture 7b and flap 7g. When the flap 7g opens inward relative to the page, the inlet valve 7 is opened and enables flow through the aperture 7b toward the upper side of the aperture 7b, in a direction that is generally away from each of the attachment lines in the connection 7d. As shown in FIG. 4B, the bottom attachment line in the connection 7d can be oriented toward a distal end of the cardiac assist device, such that when the inlet valve 7 opens inward relative to the page, fluid can be directed to flow toward the outflow element 4.

As another example, FIGS. 10A-10E illustrate an example arrangement of inlet valves 7 in a cardiac assist device, where an inlet valve 7 includes a flag 7g that is connected to the cup wall via an “L” shaped connection 7d extending around two sides (two right sides) of the aperture 7b and flap 7g. When the flap 7g opens inward relative to the page, the inlet valve 7 is opened (FIG. 10E) and enables flow through the aperture 7b toward the left side of the aperture 7b, in a direction that is generally away from the attachment lines in the connection 7d.

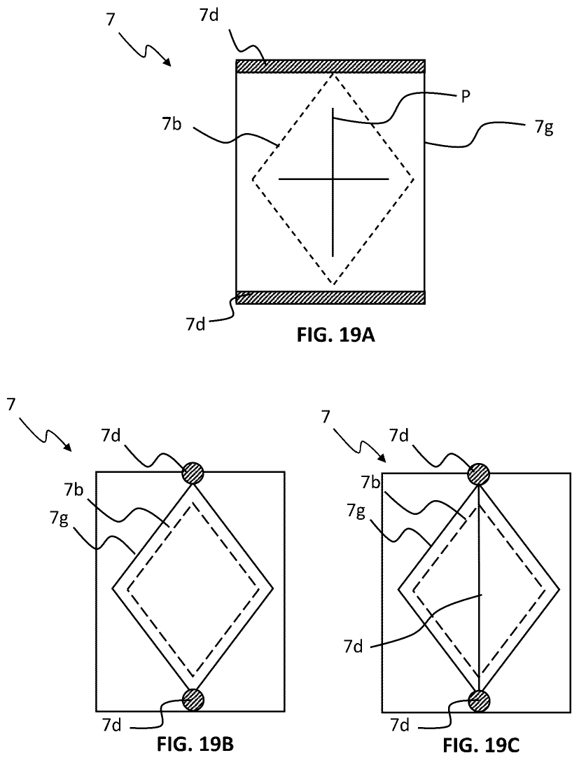

The connection 7d that attaches the flap 7g to the cup wall can be generally continuous (e.g., a line), or discontinuous (e.g., located at a series of points or other discrete regions around the flap 7g). For example, a discontinuous connection 7d can include multiple connection regions on opposite or otherwise non-adjacent portions of the flap 7g (top and bottom regions in the orientation shown in FIG. 19A). These discrete connection regions can form multiple respective hinge portions of the flap 7g that can move between closed and open states. FIG. 19A illustrates an example inlet valve 7 including a flap 7g and a connection 7d that extends along two opposite sides of the flap 7g. In this example, the flap 7g can include a perforation P that overlies the aperture 7b. In the closed state of the inlet valve 7, the perforation P is closed to prevent fluid from entering the cup. In the open state of the inlet valve 7, the perforation P can bulge open to enable fluid to pass through the perforation P and into the cup. Although the perforation P shown in FIG. 19A is cross-shaped, it should be understood that in other embodiments, the perforation P can be any suitable shape (e.g., a slit).

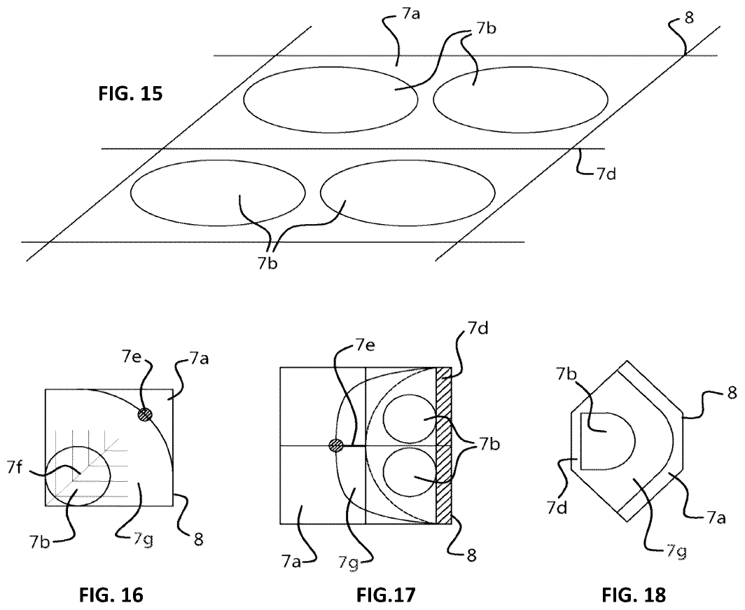

FIG. 19B illustrates another example inlet valve 7 including a flap 7g and a connection 7d that attaches the flap 7g to the cup wall, where the connection 7d includes connection points at opposite vertices of the flap 7g (top and bottom vertices in the orientation shown in FIG. 19B). In this example, the flap 7g is diamond-shaped and generally aligned with a diamond-shaped aperture 7b. In the closed state of the inlet valve 7, the flap 7g lies against the cup wall, thereby covering the aperture 7b to prevent fluid from entering the cup. In the open state of the inlet valve 7, the free end portions of the flap 7g (left and right portions as shown in FIG. 19B) can move away from the cup wall, thereby enabling fluid to pass into the cup. FIG. 19C illustrates an example inlet valve 7 similar to FIG. 19B, except the inlet valve 7 shown in FIG. 19C further includes a connection line region of connection 7d that joins the upper and lower connection points to form a “butterfly”-type valve. Although the flaps 2g and apertures 7b shown in FIGS. 19B and 19C are diamond-shaped, it should be understood that the flap 2g, the aperture 7b, or both can be any suitable shape (e.g., diamond-shaped flap 2g covering a circular aperture 7b). Other examples of “butterfly”-type valves are described herein, such as that depicted in FIG. 15.

The cup wall 2a can include a coaptation area at a perimeter portion of the aperture 7b. The flap 7g can have an open position in which the flap allows movement of fluid into the cup via the aperture 7b and a closed position in which the flap reduces movement of fluid out of the cup via the aperture. For example, the flap 7g can be configured to contact the cup 2 at the coaptation area to reduce movement of fluid out of the cup via the aperture 7b while the balloon 5 moves from the first state toward the second state. The flap 7g can also be configured to move away from the cup at the coaptation area to allow movement of fluid into the cup via the aperture 7b while the balloon 5 moves from the second state toward the first state. The coaptation area can include a portion of the cup wall 2a (e.g., a portion of the outer layer 7a) between an inner edge of the struts at a given interstice and an outer edge of the aperture 7b. Furthermore, the coaptation area can be entirely within the interstice, extend beyond the interstice around all of a perimeter of the aperture 7b, or extend beyond the interstice around some (e.g., at least 50%) of the perimeter of the aperture 7b.

The flap 7g is indicated in FIG. 3 as having a coaptation length C. In at least some embodiments, the coaptation length C is at least 0.5 mm, such as within a range from 0.5 mm to 5 mm. The length between adjacent struts of the skeleton structure 8 is indicated as S, and the distance between a strut and the aperture 7b (having a diameter D) as L. In a particular example, when the coaptation length C is at least 2 mm, a proper internal flow of the fluid can be achieved. Furthermore, it can be advantageous for surfaces of the cup wall 2a and the flap 7g intended to be in contact with fluid during operation of the cardiac assist device to be smooth and/or lubricious, such as to reduce friction with the fluid entering the inner volume of the cup 2.

The distance between (i) the inlet valve aperture 7b (e.g., center of the inlet valve aperture 7b) and (ii) a hinge portion of the flap located at the edge of the connection 7d is indicated in FIG. 3 as a distance R, which operates as a swing radius of the flap 7g. Generally, shorter distances of R enable the flap 7g to travel shorter arcuate distances when transitioning from an open state to a closed state, thereby advantageously enabling the inlet valve 7 to have a quicker response time when the device is transitioning from the filling operational mode to the pumping operational mode). This can, for example, enable the cardiac assist device to be more efficient in pumping fluid that it has received and contained in the cup 2. In some examples, the distance R can be between about 1 mm and about 3 mm.

As described herein, one or more inlet valves 7 can be arranged in the cup wall 2a. In some embodiments, multiple inlet valves 7 can be arranged in various patterns in the cup wall 2a, with their respective flaps 7g and connections 7d correspondingly oriented to achieve a particular desired fluid flow pattern.

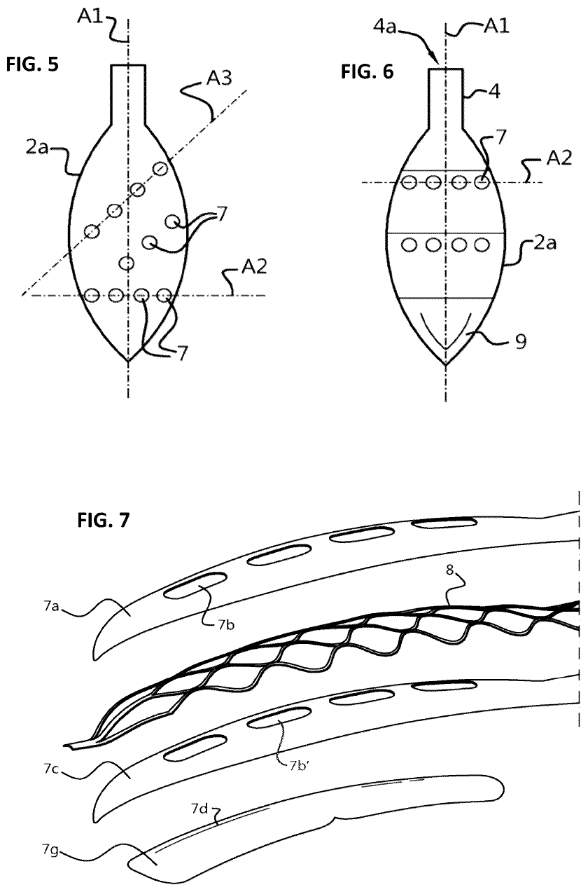

For example, FIG. 5 is a side view of a cardiac assist device in accordance with at least some embodiments of the present technology having various directions of lines of inlet valves. The apertures 7b in the cup wall 2a may be implemented as slits in the surface of the cup wall 2a (e.g., in a surface of the outer layer 7a). As an alternative, the apertures 7b may be implemented as a series of apertures 7b, e.g. in a straight line. In some embodiments (e.g., as shown in FIG. 1A) the inlet valves 7 are arranged in a pattern of one or more longitudinal lines along the cup wall 2a, such as parallel to the longitudinal axis A1 or within a range of 10 degrees from parallel to the longitudinal axis A1. In addition or alternatively, the inlet valves 7 can be arranged in a pattern of one or more circumferential lines along the cup wall 2a, such as in a direction A2 as shown in FIG. 5 perpendicular to the longitudinal axis A1 or within a range of 10 degrees from parallel to the longitudinal axis A1. Combining these examples, the inlet valves 7 can be arranged in a staggered or checkerboard-like pattern of perpendicular lines with apertures 7b. The inlet valves 7 can also be arranged in a pattern of one or more helical lines along the cup wall 2a, such as in a direction A3 shown in FIG. 5. Such helical lines can have various angles with respect to the longitudinal axis A1. In some embodiments, a single screw-thread-like line with apertures 7b is provided over a major part of the cup wall 2a. Furthermore, multiple lines can be spaced apart from one another along the longitudinal axis A1 and/or circumferentially around the longitudinal axis A1.

Multiple inlet valve 7 lines may be provided in parallel along the longitudinal direction A1 of the cup wall 2a. This can increase directional and/or spiral flow inside the cup 2. The inlet valve apertures 7 and flaps 7g may furthermore have an orientation in the same direction. Fluid flow over the flaps 7g in this manner can help to close the inlet valves 7 faster. For example, inflow from a given one of the inlet valves 7 can be directed against the flap 7g of an adjacent inlet valve 7 to facilitate closing of the adjacent inlet valve 7. In at least some cases, the flaps 7g extend from the cup wall 2a proximally and/or in the same circumferential direction relative to the longitudinal axis A1 to direct fluid toward the outflow element 4a directly or in spiral flow pattern.

FIG. 6 is a side view of a cardiac assist device in accordance with at least some embodiments of the present technology. As shown in FIG. 6, the cardiac assist device can include a distal one-way valve 9 arranged in the cup wall 2a remote from the outflow element 4. The distal one-way valve 9 can take the place of or be present in addition to other one-way inlet valves, such as one-way inlet valves at the two circumferential lines with apertures 7b shown in FIG. 6. The distal one-way valve 9 can be useful, for example, to enhance circulation and throughput of fluid during pumping operations of the cardiac assist device, such as in a distal end part of the cup wall 2a. For these and/or other reasons, the distal one-way valve 9 can reduce thrombogenicity. The distal one-way valve 9 can be, for example, a cone valve, a reversed nozzle valve, and/or a valve balloon. When the distal one-way valve 9 is a valve balloon and in other cases, the distal one-way valve 9 can act as an atraumatic bumper and/or positioning aid.

FIG. 7 is an exploded perspective view of a series of inlet valves of an example cardiac assist device in accordance with at least some embodiments of the present technology. With reference to FIG. 7, the cup wall 2a (partially shown in FIG. 7) can include the skeleton structure 8 (partially shown in FIG. 7), the outer layer 7a provided with the inlet valve apertures 7b, and the inner layer 7c (with corresponding inlet valve apertures 7b′). The combination of the skeleton structure 8 with the outer layer 7a and the inner layer 7c can, for example, be referred to as an encapsulated membrane structure. The flap 7g can be connected to the cup wall 2a via the inner layer 7c along the connection 7d. The flap(s) 7g can be positioned to perform closure of several of the inflow apertures 7b, 7b′. For example, flap(s) 7g can be coupled to the cup wall 2a with connection 7d oriented longitudinally so as to be parallel to the longitudinal axis of the cup 2. Additionally or alternatively, similar to that described respect to FIGS. 5 and 7, at least some of the inflow apertures 7b, 7b′ can be oriented at an oblique angle relative to the longitudinal axis, or in some examples, in a spiral direction around the longitudinal axis and the flap(s) 7g can be similarly oriented to perform closure of such apertures 7b, 7b′. In any of these configurations, the positioning of inlet valve apertures 7b, 7b′ and the orientation of flap(s) 7g can be selected so that the flap may cover and close a plurality of inlet valve apertures 7b, 7b′ simultaneously. In some examples, multiple flaps 7g may be arranged generally parallel to each other (and parallel to a longitudinal axis of the cup 2) around the circumference of the cup 2, each flap being configured to close a plurality of inlet valve apertures 7b, 7b′ arranged in general alignment with a longitudinal axis of the flap. The coaptation area thus formed, for example, can be between 0.5 and 5 mm, e.g. between 2 and 3 mm. The inlet valve apertures 7b can be at least 0.5 mm in diameter, e.g. at least 3.5 mm. The orientations of the coaptation areas, of the connections 7d, and/or of the flaps 7g relative to the inflow areas (inlet valve apertures) can promote directional inflow of fluid inside the cup 2, (e.g., cause a spiral flow within the cup 2 or flow directed toward the outflow element 4, or as further described elsewhere herein). When multiple flaps 7g are present, a spiral flow within the cup 2 can cause adjacent inlet valves 7 to close in an efficient cascading manner.

FIGS. 7A-7C are top plan views of different arrangements of inlet valves 7 of cardiac assist devices in accordance with at least some embodiments of the present technology. The cup wall 2a can comprise a skeleton structure 8 with a regular opening pattern, wherein the inlet valves 7 are aligned with the regular interstice or opening pattern. The regular opening pattern can, for example, define diamond shaped or hexagonal shaped openings. The skeleton structure 8 can be a metal (e.g., nitinol) cage which allows elongation of the cardiac assist device to make the outer diameter smaller to allow transport through or in a catheter. As shown, the inlet valves 7 can be arranged in a line parallel to the longitudinal axis A1 (FIG. 7A), perpendicular to the longitudinal axis A1 (FIG. 7B), and/or in a direction A3 at an angle to the longitudinal axis A1 (FIG. 7C).

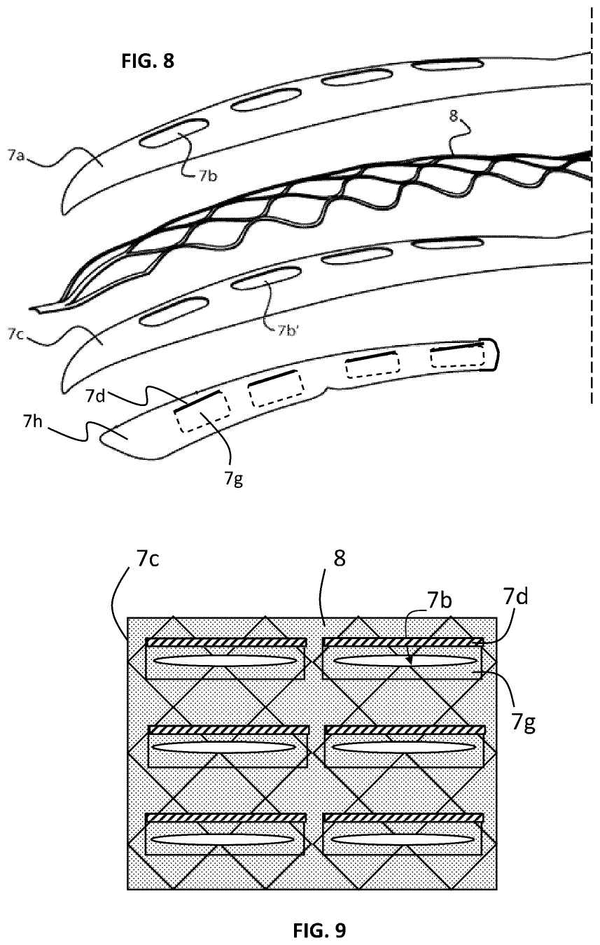

FIG. 8 is an exploded perspective view of inlet valves of an example cardiac assist device in accordance with at least some embodiments of the present technology. The example shown in FIG. 8 is similar to that described above with respect to FIG. 7, except the example shown in FIG. 8 includes the second inner layer 7h having flaps 7g. As shown in FIG. 8, the cup wall can include the skeleton structure 8, an outer layer 7a provided with inlet valve apertures 7b, a first inner layer 7c provided with corresponding inlet valve aperture 7b′, and a second inner layer 7h defining flaps 7g corresponding to the inlet valve apertures 7b, 7b′. The flaps 7g can be integrally joined with, or coupled to, the second inner layer 7h at respective connections 7d. Furthermore, the orientations of the coaptation areas, of the connection points 7d, and/or the flaps 7g relative to the inflow areas (inlet valve apertures) can promote directional inflow of fluid inside the cup 2, as further described elsewhere herein.

FIG. 9 partially illustrates an example embodiment of an inlet valve arrangement in which the inlet valve apertures 7b in one or more layers of the cup wall (e.g., outer layer and/or inner layer on either side of the skeleton structure 8). The arrangement shown in FIG. 9 can be similar to that described above with respect to FIG. 8, in that the inlet valve apertures 7b can be formed in the inner layer 7c of the cup wall 2a and covered by respective elongated flaps 7g formed in a second inner layer that can be similar to second inner layer 7h as shown in FIG. 8. However, in the embodiment shown in FIG. 9, the inlet valve apertures 7b are formed as longitudinal slits that are generally parallel to the longitudinal axis of the cardiac assist device (e.g., longitudinal line A1 shown in FIGS. 5 and 6). Each of the longitudinal slits can extend across and/or otherwise overlap with two or more open cells of the skeleton structure 8, thereby providing a greater effective inflow surface area (e.g., combined area of the inlet valve apertures 7b) with a low pressure gradient across the cup wall. Additionally, because of the shorter length of the swing radius of the flaps 7g (measured orthogonal to the attachment line at connection 7d), the inlet valve arrangement shown in FIG. 9 can accordingly have an improved, faster inlet valve closure time. Furthermore, the elongated shape of the inlet valve apertures 7b may provide a more homogeneous inflow area, advantageously providing for a more linear flow path of fluid through the inlet valve 7 and into the cup 2.

FIGS. 10A-10E partially illustrate an example embodiment of an inlet valve arrangement in which the inlet valve apertures 7b are formed in one or more layers of the cup wall (e.g., outer layer 7a and/or inner layer 7c on either side of the skeleton structure 8). The combination of the skeleton structure 8 with the outer layer 7a and the inner layer 7c can, for example, be referred to as an encapsulated membrane structure (FIG. 10B). One of more of the inlet valve apertures 7b can each overlap with a respective open cell of the skeleton structure 8. The arrangement shown in FIG. 9 can be similar to that described above with respect to FIG. 8, in that the inlet valve apertures 7b can be formed in the inner layer 7c of the cup wall 2a and covered by respective flaps 2g formed in a second inner layer 7h (FIG. 10C). However, in the embodiment shown in FIGS. 10A-10E, all (or a significant portion, such as a majority) of the flaps 7g are formed in the second inner layer 7h. For example, the second inner layer 2h can include an array of flaps 7g that extend in both a radial (circumferential) direction around the skeleton structure 8 and a longitudinal direction along the skeleton structure 8. As shown in FIG. 10C, the flaps 7g can have a connection 7d that extends along attachment lines at two right sides of the inlet valve apertures 7b (or flaps 7g). In this example, when the flap 7g opens inward relative to the page such as in the filling operational mode of the device, the inlet valve 7 is opened (FIG. 10E) and enables flow through the aperture 7b toward the left side of the aperture 7b, in a direction that is generally away from the attachment lines in the connection 7d. The flap 7g can also close to close the inlet valve 7 (FIG. 10D), such as in the pumping operational mode of the device.

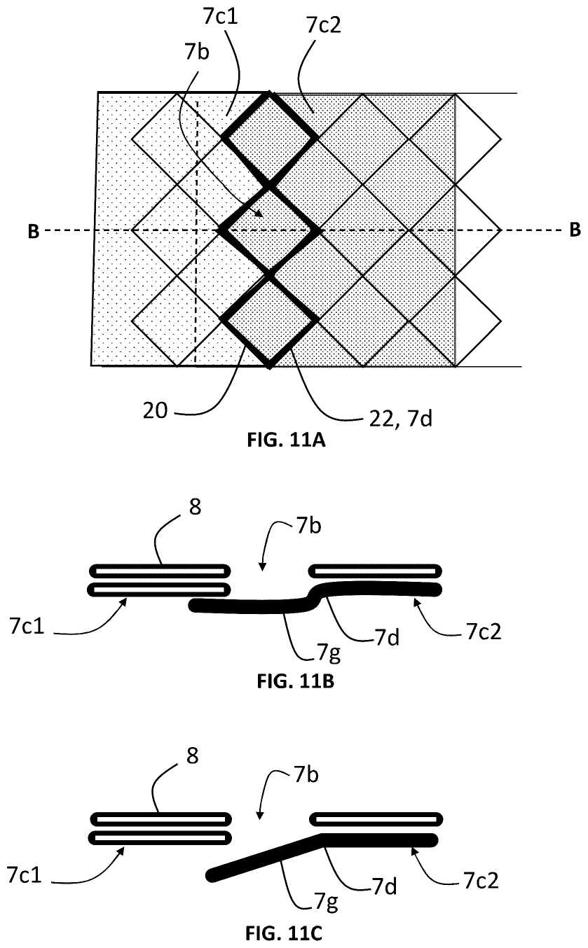

FIGS. 11A-11C partially illustrate an example embodiment of an inlet valve arrangement in which flap(s) of one or more inlet valves are formed by a circumferential (or partially circumferential) region of overlapping membranes within or adjacent the cup wall. For example, as shown in FIGS. 11A-11C, the cup wall can include a first inner layer 7c1 and a second inner layer 7c2. The first inner layer 7c1 can cover a first longitudinal portion of the skeleton structure 8 up to a first side 20 (e.g., proximal edge) of a particular inlet valve-designed row of open cells (functioning as inlet valve apertures 7b). The first inner layer 7c1 can be attached to the skeleton structure 8 at the first side 20 of the inlet valve-designated row of open cells. Meanwhile, the second inner layer 7c2 can cover a second longitudinal portion of the skeleton structure 8 up to a second side 22 (e.g., distal edge) of the same row of open cells, thereby leaving the row of valve-designated open cells uncovered. The second inner layer 7c2 can be attached to the skeleton structure at the second side 22 of the inlet valve-designated row of open cells, but extend farther to overlap with a longitudinal portion of the first inner layer 7c1 at a circumferential overlapping region. This circumferential overlapping region can form a flap 7g of at least one inlet valve 7 that can move between a closed state (FIG. 11B) and an open state (FIG. 11C). In some embodiments, the circumferential overlapping region forms a single ring-shaped flap configured to cover multiple apertures 7b (e.g., a circumferential ring of open cells). Alternatively, in some embodiments, the overlapping region is partially circumferential and forms an arcuate, partial ring-shaped flap configured to cover one or more apertures 7b (e.g., a partial ring of open cells). In some embodiments, a cardiac assist device can include one or more ring-shaped flaps 7g and/or partial ring-shaped flaps 7g along its longitudinal length. Furthermore, although the example of FIGS. 11A-11C is described above primarily as including a longitudinally-oriented flap 7g formed by the first and second inner layers 7c1 and 7c2 overlapping in a longitudinal direction, it should be understood that additionally or alternatively, a cardiac assist device can include a flap 7g oriented laterally (or circumferentially) via first and second inner layers 7c1 and 7c2 similarly overlapping in a circumferential direction. Furthermore, the direction or angle of overlapping inner layers 7c1 and 7c2 can define any suitable flap orientation (e.g., flap(s) 7g oriented at a non-orthogonal angle to the longitudinal axis, such as 45 degrees from the longitudinal axis), which can, for example, form a helical arrangement of inlet valves formed by an angled overlapping region of the inner layers 7c1 and 7c2.

In at least some embodiments, the flap 7g is pre-shaped to a curvature of the cup wall 2a. For example, the flap 7g can have a resting curvature matching the curvature of the cup wall 2a at a given one of the interstices. The cup 2 can have a concave inner surface at the given interstice and the flap 7g can have a convex outer surface at the given interstice. The outer surface of the flap 7g can contact the inner surface of the cup 2 at the coaptation area while the balloon 5 moves from the first state toward the second state. The congruent fit can enhance sealing against the cup wall 2a. This feature may also be advantageous in providing a quicker closing time. In addition or alternatively, the flap 7g can be biased toward a closed position. For example, the flap 7g can resiliently return to a state in which it contacts a corresponding coaptation area in the absence of a pressure differential that draws fluid into the cup 2.

The flap 7g can be reinforced. This can cause the flap 7g to be more resistant to deforming in response to high pressures within the cup. For example, the reinforced flap 7g may resist bulging at the aperture 7b, which can adversely affect sealing and cycling speed. Moreover, reinforcement can cause the flap 7g to be more resistant to damage from repeated motion and bending, thereby increasing the durability of the one-way valve 7 and of the overall cardiac assist device. Even further, in some embodiments, the reinforcement can impart spring-like behavior to the flap 7g for shorter response time for inlet valve closure, thereby enabling the flap 7g to return the inlet valve to its closed state more quickly before or when the balloon is being inflated in the pumping operational mode. This faster closure response time for the flap(s) 7g in the cardiac assist device reduces the amount of fluid in the cup that reverts through the inlet valve(s), which can help increase the pumping efficiency of the cardiac assist device.

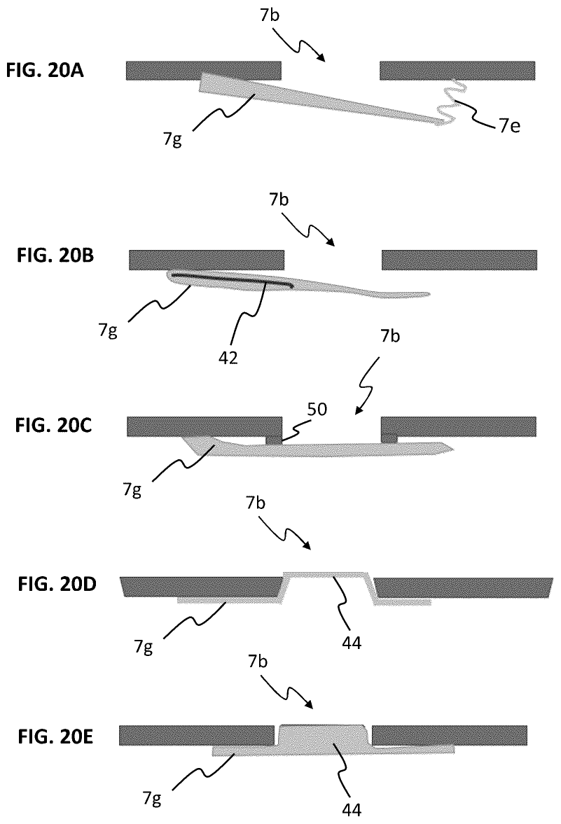

In some embodiments, the flap 7g can be reinforced with a localized thicker portion and/or primarily include a stiff material (e.g., stiffer than a second inner layer 7h to which the flap 7g is connected, and that lines the cup wall 2a). For example, as shown in FIGS. 20A and 20B, the flap 7g can have a tapering thickness (e.g., thicker closer to the connection 7d, and thinner farther from the connection 7d).

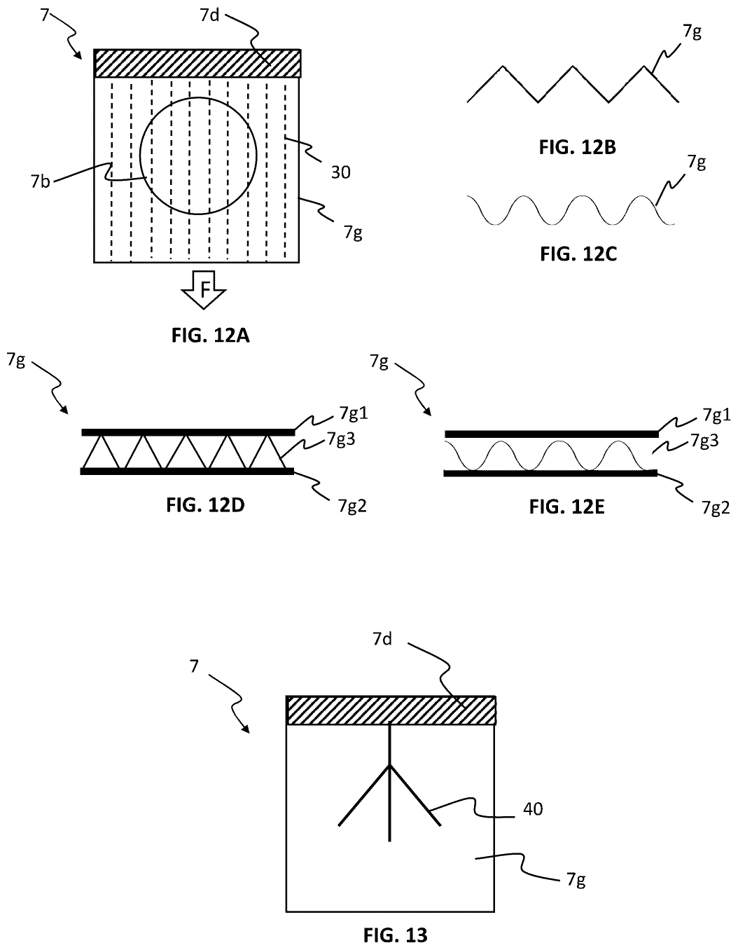

In some embodiments, a reinforcement in the flap 7g, the flap 7g can additionally or alternatively include at least one fold oriented along the flap in a direction generally aligned with the direction of fluid inflow. For example, as shown in FIG. 12A, the flap 7g can include a series of one or more folds (e.g., in an accordion style) directed along the direction of inflow F through the inlet valve. Such folds provide additional resilience to the flap 7g against bending, etc. during opening and closure of the flap 7g, and/or impart spring-like behavior to the flap 7g with shorter closure response time for inlet valve closure as described above. The fold pattern can include, for example, a triangular wave fold (FIG. 12B), sinusoidal fold (FIG. 12C), and/or other suitable fold (e.g., pleating). Additionally or alternatively, in some embodiments, the flap can include multiple layers including a folded reinforcement layer. For example, as shown in FIGS. 12D and 12E, the flap 7g can include an outer flap layer 7g1, an inner flap layer 7g2, and a folded reinforcement layer 7g3 between the outer flap layer 7g1 and the inner flap layer 7g2. The folded reinforcement layer can, similar to that described above with respect to FIGS. 12A-12C, include a series of one or more folds directed along the direction of inflow through the inlet valve, and/or can include any suitable fold pattern such as a triangular wave fold (FIG. 12D) or a sinusoidal fold (FIG. 12E).

Alternatively or in addition, the flap 7g can include an embedded or applied reinforcement structure. The reinforcement structure can, in some embodiments, provide a hinge point for flap movement and/or impart spring-like behavior to the flap 7g with shorter closure response time for inlet valve closure. In some embodiments, at least a portion of the reinforcement structure can be aligned with the direction of fluid inflow through the inlet valve and/or extending away from the connection 7d for the flap 7g. This reinforcement structure can be made of a material different than a primary material of the flap 7g. For example, the flap 7g can be primarily polymeric and the reinforcement structure can be metallic, such as nitinol. As another example, the flap 7g can primarily comprise a first polymeric material and the reinforcement structure can comprise a second, denser polymeric material, such as ultra-high molecular weight polyethylene (e.g., DYNEEMA®). The reinforcement can be thin, e.g. wire shaped, and/or be branched as shown in FIG. 13. In some embodiments, as shown in FIG. 13, the reinforcement structure can be an extension 40 (e.g., branched or feathered extension) that is integrally formed with or coupled to a portion of the cup wall, such as the skeleton structure 8. The extension 40 can, for example, at least partially lie along a surface of the flap 7g (e.g., inner or outer face of the flap 7g), and/or be at least partially embedded within the body of the flap 7g. As another example, in some embodiments such as that shown in FIG. 20B, the reinforcement structure can include an embedded member 42 forming a spine along at least a portion of the flap length. Further example details regarding reinforcement of the flap 7g are provided below with reference to FIG. 16.

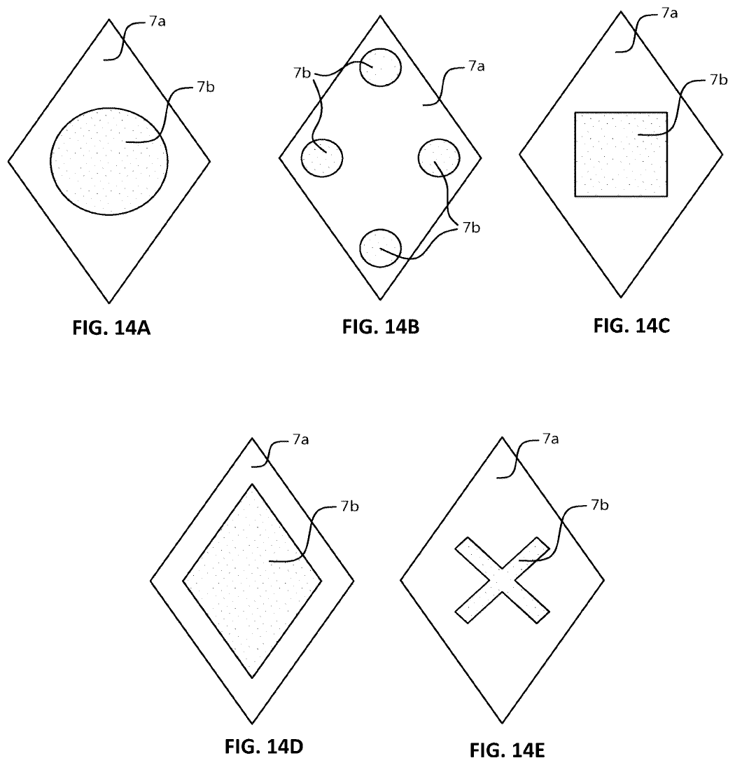

FIGS. 14A-14E are top plan views of different apertures of one-way inlet valves of cardiac assist devices in accordance with at least some embodiments of the present technology. FIG. 14A shows a cup wall 2a (e.g., an outer layer 7a of a cup wall 2a) provided with a round aperture 7b, which may provide strong resistance against tearing of the outer layer 7a. FIG. 14B shows multiple (four) round apertures 7b spaced apart from one another. Thus, more than one aperture 7b (e.g., two, three, four, or five apertures 7b) can be present at the same interstice. Multiple spaced-apart apertures at the same interstice may allow for even further tear resistance, flap-bulging resistance, and/or possibly a larger total inflow opening area. FIG. 14C shows an aperture 7b having a rectangular (e.g., square) shape, which may facilitate manufacturing (e.g. by stamping of the outer layer 7a). FIG. 14D shows a diamond-shaped aperture 7b congruent to the shape of the openings of the skeleton structure 8 as shown in FIGS. 7A-7C. FIG. 14E shows a cross-shaped aperture 7b. The apertures 7b, for example, can include two or more intersecting incisions in the cup wall 2a (e.g., in the outer layer 7a of the cup wall). In at least some cases, forming the aperture 7b from incisions may facilitate quicker and/or more efficient closing of the inlet valve 7. The outer layer 7a of the inlet valve 7 can comprise an aperture 7b which is congruent with an opening in the regular opening pattern, e.g. a diamond or hexagon shape. Furthermore, the apertures 7b can be ellipsoid, diamond-shaped, circular, rectangular, cross-shaped or have another suitable form.

FIG. 15 is a schematic perspective view of portions of a one-way inlet valve of a cardiac assist device in accordance with at least some embodiments of the present technology. Four round apertures 7b are provided in the cup wall 2a (e.g., in the outer layer 7a of the cup wall) between struts of the skeleton structure 8. The connection 7d of the flap 7g (not shown for clarity) can include a small line in the middle running parallel to and along two of the four apertures 7b, e.g. obtained by micro-welding techniques. By attaching the flap 7g in this manner to the cup wall 2a (e.g., to the outer layer 7a of the cup wall 2a), a butterfly type of one-way valve 7 may be created, wherein each side of the flap 7g is arranged to seal off two of the four apertures 7b. This configuration of apertures 7b can enhance inflow capacity and/or reduce cup implosion effects. Also, when a part of the inlet valves 7 is blocked in the ventricle, additional capacity can be particularly advantageous, such as to reduce negative pressure and hemolysis. This design may also speed up valve closure and/or lower leakage flow. For example, smaller movement of the flap 7g can cause the one-way valve 7 to close faster.

FIG. 16 shows a top view of a one-way valve 7 in accordance with at least some embodiments of the present technology, wherein the aperture 7b is positioned at a corner of the outer layer 7a spanning a rectangular opening in the skeleton structure 8. The flap 7g has a rounded edge away from the aperture 7b. Also, the flap 7g is provided with a reinforcement structure 7f in the form of branch like thickenings of the flap 7g. In at least some cases, the flap 7g includes a hinge portion at which the flap 7g is hingedly connected to the cup wall 2a. The flap 7g can further include a contact portion configured to contact the cup wall 2a at the coaptation area to reduce movement of fluid out of the cup via the aperture 7b while the balloon 5 moves from the first state toward the second state. The flap 7g can also include a central portion between the contact portion and the hinge portion. The central portion of the flap 7g can include a reinforcing spine alone or as part of a more complex reinforcement structure 7f. For example, the reinforcement structure 7f can include reinforcing branches extending from the spine toward the contact portion of the flap 7g. A composition of the contact portion of the flap 7g can be different than a composition of the spine. For example, the contact portion of the flap 7g can be at least primarily polymeric, and the spine can be at least primarily metallic. Furthermore, an average thickness of the central portion of the flap 7g can be greater than an average thickness of the contact portion of the flap 7g. Due to this thickness difference, the presence or the reinforcement structure 7f, or for another reason, an average stiffness of the central portion of the flap 7g can be greater than an average stiffness of the contact portion of the flap 7g. This can be advantageous, for example, to promote sealing efficiency and reduce or prevent deformation of the flap 7g in response to high pressure within the cup.

In some embodiments, as shown for example in FIGS. 16, 17, and 20A, the flap 7g can be further attached to the cup wall 2a via at least one anchor element 7e. The anchor element 7e can limit an available opening distance of the flap 7g relative to the cup wall 2a. This can be useful, for example, to reduce the time needed to return the flap 7g to a closed sealing position. Similar to that described above in relation to flap reinforcements, such faster closure response time for the flap(s) 7g in the cardiac assist device reduces the amount of fluid in the cup that reverts through the inlet valve(s), which can help increase the pumping efficiency of the cardiac assist device. In addition or alternatively, the anchor element 7e can be useful to reduce or prevent bulging of the flap 7g from the aperture 7b in response to pressure within the cup 2.

In at least some embodiments, the anchor element 7e can be located at an end portion of the flap 7g that is distanced or spaced apart from its hinge portion (e.g., distanced or spaced apart from the connection 7d where the flap 7g is attached to the cup wall 2a. For example, the end portion can be opposite from the connection 7d. The anchor element 7e can be located between the cup wall 2a and the end portion of the flap 7g. For example, the hinge portion of the flap 7g can be at one side of the aperture 7b and the anchor element 7e can be at an opposite side of the aperture 7b. The anchor element 7e can include any suitable connection and/or structure that restricts movement of the end portion of the flap 7g away from the cup wall 2a while the balloon 5 moves from the second state toward the first state. For example, the anchor element 7e can include a weld (e.g., a spot weld) that connects the end portion of the flap 7g to the cup wall 2a at a particular location, thereby restricting movement of the end portion of the flap 7g at the location of the weld. As another example, as shown in FIG. 20A, the anchor element 7e can include a spring or a tether with a predetermined length that limits the opening distance between the end portion of the flap 7g and the cup wall 2a. In some embodiments having a spring or spring-like anchor element 7e (e.g., functioning as a tension spring), the anchor element 7e can help bias the flap 7g toward the closed state, thereby further reducing closure response time for the flap 7g.

FIG. 17 shows a top view of an example inlet valve 7 including at least one anchor element 7e in accordance with at least some embodiments of the present technology, wherein the outer layer 7a is provided with two apertures 7b within two adjacent openings in the skeleton structure 8. The two apertures 7b are positioned off-center in the outer layer 7a spanning the two adjacent openings in the skeleton structure 8. The outer layer 7a also spans two or more further neighboring openings in the skeleton structure 8 where no apertures 7b are present. The flap 7g is arranged to seal off the two apertures 7b when lying against the outer layer 7a. The connection 7d can be next to the two apertures 7b. An anchor element 7e extends from an apex of the flap 7g to the center of the struts of the skeleton structure 8. This can shorten the closing time of the inlet valve 7 by limiting the associated travel of the flap 7g.

FIG. 18 shows a top view of an example inlet valve 7 in accordance with at least some embodiments of the present technology, wherein the skeleton structure 8 has openings in a hexagonal shape. The aperture 7b in the cup wall 2a (e.g., in the outer layer 7a of the cup wall) has a rounded shape at a part remote from the connection 7d. The flap 7g has a rounded shape as well, which can facilitate sealing of the aperture 7b in the pumping operational mode.

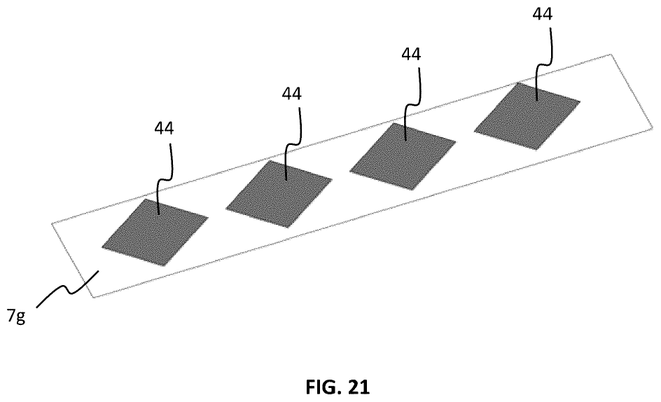

In some embodiments, an inlet valve 7 can additionally or alternatively include further textural and/or other structural features for improving sealing of the inlet valve 7 in its closed state. For example, as shown in FIG. 20C, in some embodiments, the aperture 7b can have a raised periphery forming a rim 50 configured to seal against the flap 7g. In some of these embodiments, the flap 7g can further include a complementary feature that receives or otherwise mates with the rim 50 to further improve the seal where the rim 50 and the flap 7g contact each other. Additionally or alternatively, the rim 50 and/or the portion of the flap 7g contacting the rim 50 can include a suitable fluid sealing material (e.g., an elastomeric material). As another example, as shown in FIGS. 20D and 20E, the flap 7g can include a plug 44 shaped and sized to fit into and fill the aperture 7b in a complementary (e.g., mating) manner. The plug 44 can be a pre-shaped bent portion (e.g., an embossed, molded, or otherwise raised portion) of the flap 7g (FIG. 20D), or can be a solid filled plug (FIG. 20E). Like the example shown in FIG. 20C, the plug 44 and/or the periphery of the aperture 7b contacting the plug 44 can include a suitable fluid sealing material (e.g., an elastomeric material). In some embodiments, a single flap 7g can include any suitable combination of one or more kinds of such textural features configured to improve sealing against multiple apertures 7b of multiple inlet valves. For example, as shown in FIG. 21, a flap 7g can include multiple plugs 44 shaped and sized to fill respective apertures 7b in a complementary manner. Although the example shown in FIG. 21 shows a flap 7g with diamond-shaped plugs 44 (e.g., to complement diamond-shaped apertures 7b, such as those defined in a skeleton structure 8 of the cup wall 2a as described elsewhere herein), it should be understood that the plugs 44 can have any suitable shape.

In some embodiments, the balloon 5 may also be involved in the operation of the inlet valves 7. For example, the balloon 5 can comprise a multi-stage balloon assembly having at least two balloon parts. The multi-stage balloon assembly can facilitate steering the inner flow of fluid entering the inner volume via the inlet valves 7, such as due to a shape and/or material characteristics of the multi-stage balloon assembly. In at least some cases, a multi-stage balloon assembly in accordance with at least some embodiments of the present technology comprises two separate balloons and/or at least two balloon parts having different respective rigidities. When two or more balloon parts are present, one of the balloon parts can be positioned to close off one or more inlet valves 7 when the cardiac assist device is in the pumping operational mode. In addition or alternatively, the tube 6 can comprise a plurality of channels connected to the at least two balloon parts, such as to allow for independent inflation and deflation of the balloon parts.

### Examples

The following examples are included to further describe some aspects of the present technology, and should not be used to limit the scope of the technology.

Example 1. A cardiac assist device, comprising:
a cup having a cup wall defining an inner cup volume;
an outflow element connected with the cup wall and having an aperture in fluid communication with the inner cup volume for expelling a fluid during operation;
a balloon positioned inside the cup;
a lumen in fluid communication with the balloon for inflating and deflating the balloon during operation, in a pumping operational mode and in a filling operational mode, respectively; and
one or more one-way valves arranged in the cup wall to allow the fluid to flow into the cup during the filling operational mode,
wherein at least one of the one or more one-way valves comprises a layer with one or more apertures and a flap arranged to close off the one or more apertures during the pumping operational mode.

Example 2. The cardiac assist device of Example 1, wherein the cup wall comprises a fluid-impermeable material.

Example 3. The cardiac assist device of Example 1 or 2 wherein at least one of the one or more one-way valves is configured to open and close at a cycle frequency of at least about 300 times per minute.

Example 4. The cardiac assist device according to any one of Examples 1-3, wherein the layer with the one or more apertures is an integrated part of the cup wall.

Example 5. The cardiac assist device according to any one of Examples 1-4, wherein at least one of the one or more apertures comprises a flared periphery.

Example 6. The cardiac assist device according to any one of Examples 1-5, wherein at least one of the one or more apertures comprises a raised periphery configured to seal against the flap.

Example 7. The cardiac assist device according to any one of Examples 1-6, wherein the flap is attached to the layer at a connection.

Example 8. The cardiac assist device according to Example 7, wherein the flap and connection are arranged to impart a direction of flow of the fluid during the filling operational mode.

Example 9. The cardiac assist device according to Example 7 or 8, wherein the connection is arranged on three sides surrounding at least one of the apertures.

Example 10. The cardiac assist device according to any one of Examples 7-9, wherein the flap is furthermore attached to the layer by at least one anchor element.

Example 11. The cardiac assist device according to Example 10, wherein the at least one anchor element comprises at least one selected from the group consisting of a tether and a spring.

Example 12. The cardiac assist device according to any one of Examples 7-11, wherein the flap has a polygonal shape comprising a first vertex and a second vertex opposite the first vertex, wherein the connection comprises a first connection point attaching the first vertex of the flap to the layer and a second connection point attaching the second vertex of the flap to the layer.

Example 13. The cardiac assist device according to any one of Examples 1-12, wherein the flap is pre-shaped to a curvature of the cup wall.

Example 14. The cardiac assist device according to any one of Examples 1-13, wherein the flap comprises a plug configured to mate with at least one aperture.

Example 15. The cardiac assist device according to any one of Examples 1-15, wherein the flap comprises a reinforcement.

Example 16. The cardiac assist device according to Example 15, wherein the reinforcement comprises an embedded member.

Example 17. The cardiac assist device according to Example 15, wherein the cup wall comprises a skeleton structure and the reinforcement is an extension of the skeleton structure.

Example 18. The cardiac assist device according to Example 15, wherein the reinforcement comprises at least one fold in the flap.

Example 19. The cardiac assist device according to Example 15, wherein the flap comprises an inner flap layer and an outer flap layer, and wherein the reinforcement comprises a folded layer between the inner flap layer and the outer flap layer.

Example 20. The cardiac assist device according to any one of Examples 1-19, further comprising a membrane lining the cup wall, wherein the cup wall comprises the layer with one or more apertures, and wherein the membrane comprises one or more flaps of the one or more one-way valves.

Example 21. The cardiac assist device according to any one of Examples 1-20, wherein the cup wall comprises a first inner layer and a second inner layer overlapping the first inner cup wall layer at an at least partially circumferential region, wherein the circumferential region forms at least one flap of the one or more one-way valves.

Example 22. The cardiac assist device according to any one of Examples 1-21, wherein at least a portion of the one-way valves are arranged in a pattern of one or more longitudinal lines along the cup wall.

Example 23. The cardiac assist device according to any one of Examples 1-22, wherein at least a portion of the one-way valves are arranged in a pattern of one or more circumferential lines along the cup wall.

Example 24. The cardiac assist device according to any one of Examples 1-23, wherein at least a portion of the one-way valves are arranged in a pattern of one or more helical lines along the cup wall.

Example 25. The cardiac assist device according to any one of Examples 1-24, further comprising a distal one-way valve arranged in the cup wall remote from the outflow element.

Example 26. The cardiac assist device according to any one of Examples 1-25, wherein the cup wall comprises a skeleton structure with a regular opening pattern, wherein at least a portion of the one-way valves are aligned with the regular opening pattern.

Example 27. The cardiac assist device according to Example 26, wherein the layer of the one-way valve comprises an aperture that is aligned with an opening in the regular opening pattern of the skeleton structure.

Example 28. The cardiac assist device according to Example 26, wherein the layer of at least one of the one-way valves comprises one or more apertures with a circular shape, a rectangular shape, a cross shape, or a longitudinal slit shape oriented longitudinally along the cup wall.

Example 29. The cardiac assist device according to any one of Examples 1-28, wherein the total surface area of the apertures in the one or more one-way valves is at least 50 mm².

Example 30. The cardiac assist device according to any one of Examples 1-28, wherein the total surface area of the apertures in the one or more one-way valves is at least 150 mm².

Example 31. The cardiac assist device according to any one of Examples 1-28, wherein the total surface area of the apertures in the one or more one-way valves is at least 300 mm².

Example 32. The cardiac assist device according to any one of Examples 1-31, wherein total surface area of the apertures in the one or more one-way valves is at least 10% of an outer surface area of the cup wall.

Example 33. The cardiac assist device according to any one of Examples 1-31, wherein total surface area of the apertures in the one or more one-way valves is at least 25% of an outer surface area of the cup wall.

Example 34. The cardiac assist device according to any one of Examples 1-33, wherein the flap comprises a material with a durometer value lower than a durometer value of the layer.

Example 35. The cardiac assist device according to any one of Examples 1-34, wherein the inner balloon element comprises a multi-stage balloon assembly having at least two balloon parts.

Example 36. The cardiac assist device according to Example 35, wherein one of the at least two balloon parts is positioned to close off the one or more one-way valves in the pumping operational mode.

Example 37. The cardiac assist device according to Example 35 or 36, wherein the at least two balloon parts comprise a different rigidity material.

Example 38. The cardiac assist device according to any one of Examples 35-37, further comprising a tube comprising a plurality of channels connected to the at least two balloon parts.

Example 39. A cardiac assist device, comprising:
a balloon configured to move between a first state and a second state, the balloon being more inflated in the second state than in the first state;
a shell at least partially containing the balloon, wherein the shell includes struts arranged in a mesh pattern that defines interstices;
an aperture at a given one of the interstices, wherein the shell includes a coaptation area at a perimeter portion of the aperture, and wherein moving the balloon from the second state toward the first state tends to move fluid into the shell via the aperture; and
a flap carried by the shell, wherein the flap is configured to contact the shell at the coaptation area to reduce movement of fluid out of the shell via the aperture while the balloon moves from the first state toward the second state, and to move away from the shell at the coaptation area to allow movement of fluid into the shell via the aperture while the balloon moves from the second state toward the first state.

Example 40. The cardiac assist device of Example 39, wherein the coaptation area is entirely within the given interstice.

Example 41. The cardiac assist device of Example 39, wherein the coaptation area extends beyond the given interstice around at least 50% of a full perimeter of the aperture.

Example 42. The cardiac assist device of any one of Examples 39-41, wherein an average stiffness of the shell at the coaptation area is greater than an average stiffness of a portion of the flap configured to contact the shell at the coaptation area.

Example 43. The cardiac assist device of any one of Examples 39-42, wherein:
the given interstice is a first interstice;
the interstices include a second interstice and a third interstice;
the aperture is a first aperture; and
the cardiac assist device further comprises:
   a second aperture at the second interstice, wherein moving the balloon from the second state toward the first state tends to move fluid into the shell via the second aperture, and
   a third aperture at the third interstice, wherein moving the balloon from the second state toward the first state tends to move fluid into the shell via the third aperture.

Example 44. The cardiac assist device of Example 43, wherein:
the coaptation area is a first coaptation area;
the shell includes:
   a second coaptation area at a perimeter portion of the second aperture, and
   a third coaptation area at a perimeter portion of the third aperture; and
the flap is configured to contact the shell at the first, second, and third coaptation areas to reduce movement of fluid out of the shell via the first, second, and third apertures while the balloon moves from the first state toward the second state, and to move away from the shell at the first, second, and third coaptation areas to allow movement of fluid into the shell via the first, second, and third apertures while the balloon moves from the second state toward the first state.

Example 45. The cardiac assist device of Example 43, wherein:
the flap is a first flap;
the coaptation area is a first coaptation area;
the shell includes:
   a second coaptation area at a perimeter portion of the second aperture, and
   a third coaptation area at a perimeter portion of the third aperture;
the cardiac assist device further comprises:
   a second flap carried by shell, wherein the second flap is configured to contact the shell at the second coaptation area to reduce movement of fluid out of the shell via the second aperture while the balloon moves from the first state toward the second state, and to move away from the shell at the second coaptation area to allow movement of fluid into the shell via the second aperture while the balloon moves from the second state toward the first state, and
   a third flap carried by shell, wherein the third flap is configured to contact the shell at the third coaptation area to reduce movement of fluid out of the shell via the third aperture while the balloon moves from the first state toward the second state, and to move away from the shell at the third coaptation area to allow movement of fluid into the shell via the third aperture while the balloon moves from the second state toward the first state; and
the first, second, and third flaps are independently movable relative to the shell.

Example 46. The cardiac assist device of Example 45, wherein the first, second, and third flaps extend from the shell proximally.

Example 47. The cardiac assist device of Example 45 or 46, wherein:
the shell is configured to be delivered intravascularly along a delivery path;
the shell has a longitudinal axis parallel to the delivery path; and
the first, second, and third flaps extend from the shell proximally and in the same circumferential direction relative to the longitudinal axis.

Example 48. The cardiac assist device of any one of Examples 43-47, wherein:
the shell is configured to be delivered intravascularly along a delivery path;
the shell has a longitudinal axis parallel to the delivery path; and
the first, second, and third interstices are disposed along a row extending helically around the longitudinal axis.

Example 49. The cardiac assist device of any one of Examples 43-48, wherein:
the shell is configured to be delivered intravascularly along a delivery path;
the shell has a longitudinal axis parallel to the delivery path; and
the first, second, and third interstices are disposed along a row within 10 degrees of perpendicular to the longitudinal axis.

Example 50. The cardiac assist device of any one of Examples 43-49, wherein:
the shell is configured to be delivered intravascularly along a delivery path;
the shell has a longitudinal axis parallel to the delivery path;
the first, second, and third interstices are disposed along a first row;
the interstices include a fourth interstice, a fifth interstice, and a sixth interstice disposed along a second row distally offset from the first row along the longitudinal axis; and
the cardiac assist device further comprises:
   a fourth aperture at the fourth interstice, wherein moving the balloon from the second state toward the first state tends to move fluid into the shell via the fourth aperture,
   a fifth aperture at the fifth interstice, wherein moving the balloon from the second state toward the first state tends to move fluid into the shell via the fifth aperture, and
   a sixth aperture at the sixth interstice, wherein moving the balloon from the second state toward the first state tends to move fluid into the shell via the sixth aperture.

Example 51. The cardiac assist device of Example 50, wherein the first and second rows are helically disposed around the longitudinal axis.

Example 52. The cardiac assist device of Example 50 or 51, wherein the first and second rows are within 10 degrees of perpendicular to the longitudinal axis.

Example 53. The cardiac assist device of Example 50 or 51, wherein the first and second rows are within 10 degrees of parallel to the longitudinal axis.

Example 54. The cardiac assist device of any one of Examples 50-53, wherein the first row is within 10 degrees of parallel to the second row.

Example 55. The cardiac assist device of any one of Examples 50-54, wherein:
the first row is helically disposed around the longitudinal axis; and
the second row is within 10 degrees of perpendicular to the longitudinal axis.

Example 56. The cardiac assist device of any one of Examples 50-55, wherein:
the coaptation area is a first coaptation area;
the shell includes:
   a second coaptation area at a perimeter portion of the second aperture,
   a third coaptation area at a perimeter portion of the third aperture,
   a fourth coaptation area at a perimeter portion of the fourth aperture,
   a fifth coaptation area at a perimeter portion of the fifth aperture, and
   a sixth coaptation area at a perimeter portion of the sixth aperture,
the flap is a first flap configured to contact the shell at the first, second, and third coaptation areas to reduce movement of fluid out of the shell via the first, second, and third apertures while the balloon moves from the first state toward the second state, and to move away from the shell at the first, second, and third coaptation areas to allow movement of fluid into the shell via the first, second, and third apertures while the balloon moves from the second state toward the first state;
the cardiac assist device further comprises a second flap configured to contact the shell at the fourth, fifth, and sixth coaptation areas to reduce movement of fluid out of the shell via the fourth, fifth, and sixth apertures while the balloon moves from the first state toward the second state, and to move away from the shell at the fourth, fifth, and sixth coaptation areas to allow movement of fluid into the shell via the fourth, fifth, and sixth apertures while the balloon moves from the second state toward the first state; and
the first and second flaps are independently movable relative to the shell.

Example 57. The cardiac assist device of Example 56, wherein the first and second flaps extend from the shell proximally.

Example 58. The cardiac assist device of Example 56 or 57, wherein the first and second flaps extend from the shell proximally and in the same circumferential direction relative to the longitudinal axis.

Example 59. The cardiac assist device of any one of Examples 39-58, wherein:
the shell includes a membrane carried by the struts;
the membrane defines the aperture; and
the coaptation area includes a portion of the membrane between an inner edge of the struts at the given interstice and an outer edge of the aperture.

Example 60. The cardiac assist device of any one of Examples 39-59, wherein an average stiffness of the membrane at the coaptation area is greater than an average stiffness of the flap at the coaptation area.

Example 61. The cardiac assist device of any one of Examples 39-60, wherein:
the given interstice is polygonal; and
the aperture is ellipsoid.

Example 62. The cardiac assist device of any one of Examples 39-60, wherein the given interstice and the aperture are polygonal.

Example 63. The cardiac assist device of any one of Examples 39-60, wherein the given interstice and the aperture are diamond shaped.

Example 64. The cardiac assist device of any one of Examples 39-63, wherein:
the aperture is a first aperture;
the cardiac assist device further comprises a second aperture at the given interstice;
the coaptation area is at a perimeter portion of the second aperture;
moving the balloon from the second state toward the first state tends to move fluid into the shell via the second aperture; and
the first and second apertures are spaced apart from one another.

Example 65. The cardiac assist device of any one of Examples 39-64, wherein a distance between an inner edge of the struts at the given interstice and an outer edge of the aperture is within a range from 0.5 mm to 4 mm around at least 75% of a full perimeter of the aperture.

Example 66. The cardiac assist device of any one of Examples 39-65, wherein a distance between an inner edge of the struts around the given interstice and an outer edge of the aperture varies less than 50% around at least 75% of a full perimeter of the aperture.

Example 67. The cardiac assist device of any one of Examples 39-66, wherein:
the flap has an open position in which the flap allows movement of fluid into the shell via the aperture and a closed position in which the flap reduces movement of fluid out of the shell via the aperture; and
the flap is biased toward the closed position.

Example 68. The cardiac assist device of any one of Examples 39-67, wherein:
the flap includes:
   a hinge portion at which the flap is hingedly connected to the shell,
   a contact portion configured to contact the shell at the coaptation area to reduce movement of fluid out of the shell via the aperture while the balloon moves from the first state toward the second state, and
   a central portion between the contact portion and the hinge portion; and
an average stiffness of the central portion of the flap is greater than an average stiffness of the contact portion of the flap.

Example 69. The cardiac assist device of Example 68, wherein an average thickness of the central portion of the flap is greater than an average thickness of the contact portion of the flap.

Example 70. The cardiac assist device of Example 68 or 69, wherein the central portion of the flap includes a reinforcing member.

Example 71. The cardiac assist device of Example 70, wherein the central portion of the flap includes reinforcing branches extending from the reinforcing member toward the contact portion of the flap.

Example 72. The cardiac assist device of Example 70 or 71, wherein a composition of the contact portion of the flap is different than a composition of the reinforcing member.

Example 73. The cardiac assist device of Example 72, wherein the contact portion of the flap is at least primarily polymeric, and the reinforcing member is at least primarily metallic.

Example 74. The cardiac assist device of any one of Examples 39-73, wherein:
the shell has a concave inner surface at the given interstice;
the flap has a convex outer surface at the given interstice; and
the outer surface of the flap contacts the inner surface of the shell at the coaptation area while the balloon moves from the first state toward the second state.

Example 75. The cardiac assist device of any one of Examples 39-74, wherein:
the shell has a curvature at the given interstice; and
the flap has a resting curvature matching the curvature of the shell at the given interstice.

Example 76. The cardiac assist device of any one of Examples 39-75, wherein:
the flap includes:
   a hinge portion at which the flap is hingedly connected to the shell, and
   an end portion opposite to the hinge portion; and
the cardiac assist device further comprises a connection between the shell and the end portion of the flap that restricts movement of the end portion of the flap away from the shell while the balloon moves from the second state toward the first state.

Example 77. The cardiac assist device of Example 76, wherein the connection between the shell and the end portion of the flap is a weld.

Example 78. The cardiac assist device of Example 76, wherein the connection between the shell and the end portion of the flap is a tether or a spring.

Example 79. A method of providing cardiac assist, comprising:
intravascularly advancing a cardiac assist device toward a heart of a patient while the cardiac assist device is in a low-profile delivery state;
locating the cardiac assist device within a cardiovascular lumen of the patient;
moving the cardiac assist device from the delivery state to an expanded treatment state after locating the cardiac assist device within the cardiovascular lumen;
cycling a balloon of the cardiac assist device between a first state and a second state, the balloon being more inflated in the second state than in the first state, wherein the balloon is at least partially disposed within a shell of the cardiac assist device and wherein the shell includes struts arranged in a mesh pattern that defines interstices,
wherein cycling the balloon includes:
   moving the balloon from the second state toward the first state such that blood moves into the shell via an aperture of the cardiac assist device, the aperture being at a given one of the interstices, and
   moving the balloon from the first state toward the second state such that a flap carried by the shell contacts a coaptation area at a perimeter portion of the aperture thereby reducing movement of blood out of the shell via the aperture.

Example 80. The method of Examples 79, wherein cycling the balloon includes cycling the balloon at a rate within a range from 200 to 10,000 complete cycles per minute.

Example 81. The method of Example 78 or 79, wherein:
moving the balloon from the second state toward the first state includes moving the balloon from the second state toward the first state such that blood moves into the shell via first, second, and third apertures of the cardiac assist device, the first, second, and third apertures being at a first one of the interstices, a second one of the interstices, and a third one of the interstices, respectively; and
moving the balloon from the first state toward the second state includes moving the balloon from the first state toward the second state such that the flap contacts first, second, and third coaptation areas at respective perimeter portions of the first, second, and third apertures thereby reducing movement of blood out of the shell via the first, second, and third apertures.

Example 82. The method of any one of Examples 79-81, wherein moving the balloon from the second state toward the first state includes moving the balloon from the second state toward the first state while restricting movement of the flap away from the coaptation area via a weld between the shell and an end portion of the flap opposite to a hinge portion of the flap through which the flap is hingedly connected to the shell.

Example 83. The method of any one of Examples 79-81, wherein moving the balloon from the second state toward the first state includes moving the balloon from the second state toward the first state while restricting movement of the flap away from the coaptation area via a tether or a spring between the shell and an end portion of the flap opposite to a hinge portion of the flap through which the flap is hingedly connected to the shell.

Example 84. An intravascular blood pump comprising:
a chamber having at least one inlet and an outlet;
a volume displacement member within the chamber configured to move cyclically between a first state and a second state, wherein blood flows into the chamber through the inlet as the volume displacement member transitions from the second state to the first state and blood exits the chamber through the outlet as the volume displacement member transitions from the first state to the second state; and
one or more one-way valves, each one-way valve being associated with at least one inlet, wherein each one-way valve is configured to open and close at a frequency of at least 300 times per minute.

Example 85. The blood pump of Example 84, wherein the one or more one-way valves are configured to allow blood flow into the chamber through the at least one inlet at a rate of at least 30 ml/sec.

Example 86. The blood pump of Example 84, wherein the one or more one-way valves are configured to have a resistance to blood flowing through the at least one inlet of no more than 5×10⁸ kg/(m⁴s).

Example 87. The blood pump of any one of Examples 84-86, wherein the volume displacement member comprises a balloon.

Example 88. The blood pump of any one of Examples 84-87, wherein at least one of the one-way valves comprises a flap configured to transition between an open state in which the flap is configured to cover the at least one inlet, and a closed state in which the flap is configured to cover the at least one inlet.

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may be disclosed herein in the context of those embodiments, other embodiments may also exhibit such advantages, and not all embodiments need necessarily exhibit such advantages or other advantages disclosed herein to fall within the scope of the present technology. This disclosure and the associated technology can encompass other embodiments not expressly shown or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising,” “including,” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various structures. It should be understood that such terms do not denote absolute orientation. Furthermore, reference herein to “one embodiment,” “an embodiment,” or similar phrases means that a particular feature, structure, operation, or characteristic described in connection with such phrases can be included in at least one embodiment of the present technology. Thus, such phrases as used herein are not necessarily all referring to the same embodiment. Finally, it should be noted that various particular features, structures, operations, and characteristics of the embodiments described herein may be combined in any suitable manner in additional embodiments in accordance with the present technology.

We claim:

1. A cardiac assist device, comprising:
a cup having a cup wall defining an inner cup volume;
an outflow element connected with the cup wall and having an aperture in fluid communication with the inner cup volume for expelling a fluid during operation;
a balloon positioned inside the cup;
a lumen in fluid communication with the balloon for inflating and deflating the balloon during operation, in a pumping operational mode and in a filling operational mode, respectively; and
one or more one-way valves arranged in the cup wall to allow the fluid to flow into the cup during the filling operational mode,
wherein at least one of the one or more one-way valves comprises a layer with one or more apertures and a flap arranged to close off the one or more apertures during the pumping operational mode.

2. The cardiac assist device of claim 1, wherein the cup wall comprises a fluid-impermeable material.

3. The cardiac assist device of claim 1 or 2 wherein at least one of the one or more one-way valves is configured to open and close at a cycle frequency of at least about 300 times per minute.

4. The cardiac assist device according to any one of claims 1-3, wherein the layer with the one or more apertures is an integrated part of the cup wall.

5. The cardiac assist device according to any one of claims 1-4, wherein at least one of the one or more apertures comprises a flared periphery.

6. The cardiac assist device according to any one of claims 1-5, wherein at least one of the one or more apertures comprises a raised periphery configured to seal against the flap.

7. The cardiac assist device according to any one of claims 1-6, wherein the flap is attached to the layer at a connection.

8. The cardiac assist device according to claim 7, wherein the flap and connection are arranged to impart a direction of flow of the fluid during the filling operational mode.

9. The cardiac assist device according to claim 7 or 8, wherein the connection is arranged on three sides surrounding at least one of the apertures.

10. The cardiac assist device according to any one of claims 7-9, wherein the flap is furthermore attached to the layer by at least one anchor element.

11. The cardiac assist device according to claim 10, wherein the at least one anchor element comprises at least one selected from the group consisting of a tether and a spring.

12. The cardiac assist device according to any one of claims 7-11, wherein the flap has a polygonal shape comprising a first vertex and a second vertex opposite the first vertex, wherein the connection comprises a first connection point attaching the first vertex of the flap to the layer and a second connection point attaching the second vertex of the flap to the layer.

13. The cardiac assist device according to any one of claims 1-12, wherein the flap is pre-shaped to a curvature of the cup wall.

14. The cardiac assist device according to any one of claims 1-13, wherein the flap comprises a plug configured to mate with at least one aperture.

15. The cardiac assist device according to any one of claims 1-15, wherein the flap comprises a reinforcement.

16. The cardiac assist device according to claim 15, wherein the reinforcement comprises an embedded member.

17. The cardiac assist device according to claim 15, wherein the cup wall comprises a skeleton structure and the reinforcement is an extension of the skeleton structure.

18. The cardiac assist device according to claim 15, wherein the reinforcement comprises at least one fold in the flap.

19. The cardiac assist device according to claim 15, wherein the flap comprises an inner flap layer and an outer flap layer, and wherein the reinforcement comprises a folded layer between the inner flap layer and the outer flap layer.

20. The cardiac assist device according to any one of claims 1-19, further comprising a membrane lining the cup wall, wherein the cup wall comprises the layer with one or more apertures, and wherein the membrane comprises one or more flaps of the one or more one-way valves.

21. The cardiac assist device according to any one of claims 1-20, wherein the cup wall comprises a first inner layer and a second inner layer overlapping the first inner cup wall layer at an at least partially circumferential region, wherein the circumferential region forms at least one flap of the one or more one-way valves.

22. The cardiac assist device according to any one of claims 1-21, wherein at least a portion of the one-way valves are arranged in a pattern of one or more longitudinal lines along the cup wall.

23. The cardiac assist device according to any one of claims 1-22, wherein at least a portion of the one-way valves are arranged in a pattern of one or more circumferential lines along the cup wall.

24. The cardiac assist device according to any one of claims 1-23, wherein at least a portion of the one-way valves are arranged in a pattern of one or more helical lines along the cup wall.

25. The cardiac assist device according to any one of claims 1-24, further comprising a distal one-way valve arranged in the cup wall remote from the outflow element.

26. The cardiac assist device according to any one of claims 1-25, wherein the cup wall comprises a skeleton structure with a regular opening pattern, wherein at least a portion of the one-way valves are aligned with the regular opening pattern.

27. The cardiac assist device according to claim 26, wherein the layer of the one-way valve comprise an aperture that is aligned with an opening in the regular opening pattern of the skeleton structure.

28. The cardiac assist device according to claim 26, wherein the layer of at least one of the one-way valves comprises one or more apertures with a circular shape, a rectangular shape, a cross shape, or a longitudinal slit shape oriented longitudinally along the cup wall.

29. The cardiac assist device according to any one of claims 1-28, wherein the total surface area of the apertures in the one or more one-way valves is at least 50 mm².

30. The cardiac assist device according to any one of claims 1-28, wherein the total surface area of the apertures in the one or more one-way valves is at least 150 mm².

31. The cardiac assist device according to any one of claims 1-28, wherein the total surface area of the apertures in the one or more one-way valves is at least 300 mm².

32. The cardiac assist device according to any one of claims 1-31, wherein total surface area of the apertures in the one or more one-way valves is at least 10% of an outer surface area of the cup wall.

33. The cardiac assist device according to any one of claims 1-31, wherein total surface area of the apertures in the one or more one-way valves is at least 25% of an outer surface area of the cup wall.

34. The cardiac assist device according to any one of claims 1-33, wherein the flap comprises a material with a durometer value lower than a durometer value of the layer.

35. The cardiac assist device according to any one of claims 1-34, wherein the inner balloon element comprises a multi-stage balloon assembly having at least two balloon parts.

36. The cardiac assist device according to claim 35, wherein one of the at least two balloon parts is positioned to close off the one or more one-way valves in the pumping operational mode.

37. The cardiac assist device according to claim 35 or 36, wherein the at least two balloon parts comprise a different rigidity material.

38. The cardiac assist device according to any one of claims 35-37, further comprising a tube comprising a plurality of channels connected to the at least two balloon parts.

39. A cardiac assist device, comprising:
a balloon configured to move between a first state and a second state, the balloon being more inflated in the second state than in the first state;
a shell at least partially containing the balloon, wherein the shell includes struts arranged in a mesh pattern that defines interstices;
an aperture at a given one of the interstices, wherein the shell includes a coaptation area at a perimeter portion of the aperture, and wherein moving the balloon from the second state toward the first state tends to move fluid into the shell via the aperture; and
a flap carried by the shell, wherein the flap is configured to contact the shell at the coaptation area to reduce movement of fluid out of the shell via the aperture while the balloon moves from the first state toward the second state, and to move away from the shell at the coaptation area to allow movement of fluid into the shell via the aperture while the balloon moves from the second state toward the first state.

40. The cardiac assist device of claim 39, wherein the coaptation area is entirely within the given interstice.

41. The cardiac assist device of claim 39, wherein the coaptation area extends beyond the given interstice around at least 50% of a full perimeter of the aperture.

42. The cardiac assist device of any one of claims 39-41, wherein an average stiffness of the shell at the coaptation area is greater than an average stiffness of a portion of the flap configured to contact the shell at the coaptation area.

43. The cardiac assist device of any one of claims 39-42, wherein:
the given interstice is a first interstice;
the interstices include a second interstice and a third interstice;
the aperture is a first aperture; and
the cardiac assist device further comprises:
a second aperture at the second interstice, wherein moving the balloon from the second state toward the first state tends to move fluid into the shell via the second aperture, and
a third aperture at the third interstice, wherein moving the balloon from the second state toward the first state tends to move fluid into the shell via the third aperture.

44. The cardiac assist device of claim 43, wherein:
the coaptation area is a first coaptation area;
the shell includes:
a second coaptation area at a perimeter portion of the second aperture, and
a third coaptation area at a perimeter portion of the third aperture; and
the flap is configured to contact the shell at the first, second, and third coaptation areas to reduce movement of fluid out of the shell via the first, second, and third apertures while the balloon moves from the first state toward the second state, and to move away from the shell at the first, second, and third coaptation areas to allow movement of fluid into the shell via the first, second, and third apertures while the balloon moves from the second state toward the first state.

45. The cardiac assist device of claim 43, wherein:
the flap is a first flap;
the coaptation area is a first coaptation area;
the shell includes:
a second coaptation area at a perimeter portion of the second aperture, and
a third coaptation area at a perimeter portion of the third aperture;
the cardiac assist device further comprises:
a second flap carried by shell, wherein the second flap is configured to contact the shell at the second coaptation area to reduce movement of fluid out of the shell via the second aperture while the balloon moves from the first state toward the second state, and to move away from the shell at the second coaptation area to allow movement of fluid into the shell via the second aperture while the balloon moves from the second state toward the first state, and
a third flap carried by shell, wherein the third flap is configured to contact the shell at the third coaptation area to reduce movement of fluid out of the shell via the third aperture while the balloon moves from the first state toward the second state, and to move away from the shell at the third coaptation area to allow movement of fluid into the shell via the third aperture while the balloon moves from the second state toward the first state; and
the first, second, and third flaps are independently movable relative to the shell.

46. The cardiac assist device of claim 45, wherein the first, second, and third flaps extend from the shell proximally.

47. The cardiac assist device of claim 45 or 46, wherein:
the shell is configured to be delivered intravascularly along a delivery path;
the shell has a longitudinal axis parallel to the delivery path; and
the first, second, and third flaps extend from the shell proximally and in the same circumferential direction relative to the longitudinal axis.

48. The cardiac assist device of any one of claim 43-47, wherein:
the shell is configured to be delivered intravascularly along a delivery path;
the shell has a longitudinal axis parallel to the delivery path; and
the first, second, and third interstices are disposed along a row extending helically around the longitudinal axis.

49. The cardiac assist device of any one of claims 43-48, wherein:
the shell is configured to be delivered intravascularly along a delivery path;
the shell has a longitudinal axis parallel to the delivery path; and
the first, second, and third interstices are disposed along a row within 10 degrees of perpendicular to the longitudinal axis.

50. The cardiac assist device of any one of claims 43-49, wherein:
the shell is configured to be delivered intravascularly along a delivery path;
the shell has a longitudinal axis parallel to the delivery path;
the first, second, and third interstices are disposed along a first row;
the interstices include a fourth interstice, a fifth interstice, and a sixth interstice disposed along a second row distally offset from the first row along the longitudinal axis; and
the cardiac assist device further comprises:
a fourth aperture at the fourth interstice, wherein moving the balloon from the second state toward the first state tends to move fluid into the shell via the fourth aperture,
a fifth aperture at the fifth interstice, wherein moving the balloon from the second state toward the first state tends to move fluid into the shell via the fifth aperture, and
a sixth aperture at the sixth interstice, wherein moving the balloon from the second state toward the first state tends to move fluid into the shell via the sixth aperture.

51. The cardiac assist device of claim 50, wherein the first and second rows are helically disposed around the longitudinal axis.

52. The cardiac assist device of claim 50 or 51, wherein the first and second rows are within 10 degrees of perpendicular to the longitudinal axis.

53. The cardiac assist device of claim 50 or 51, wherein the first and second rows are within 10 degrees of parallel to the longitudinal axis.

54. The cardiac assist device of any one of claims 50-53, wherein the first row is within 10 degrees of parallel to the second row.

55. The cardiac assist device of any one of claims 50-54, wherein:
the first row is helically disposed around the longitudinal axis; and
the second row is within 10 degrees of perpendicular to the longitudinal axis.

56. The cardiac assist device of any one of claims 50-55, wherein:
the coaptation area is a first coaptation area;
the shell includes:
a second coaptation area at a perimeter portion of the second aperture,
a third coaptation area at a perimeter portion of the third aperture,
a fourth coaptation area at a perimeter portion of the fourth aperture,
a fifth coaptation area at a perimeter portion of the fifth aperture, and
a sixth coaptation area at a perimeter portion of the sixth aperture,
the flap is a first flap configured to contact the shell at the first, second, and third coaptation areas to reduce movement of fluid out of the shell via the first, second, and third apertures while the balloon moves from the first state toward the second state, and to move away from the shell at the first, second, and third coaptation areas to allow movement of fluid into the shell via the first, second, and third apertures while the balloon moves from the second state toward the first state;
the cardiac assist device further comprises a second flap configured to contact the shell at the fourth, fifth, and sixth coaptation areas to reduce movement of fluid out of the shell via the fourth, fifth, and sixth apertures while the balloon moves from the first state toward the second state, and to move away from the shell at the fourth, fifth, and sixth coaptation areas to allow movement of fluid into the shell via the fourth, fifth, and sixth apertures while the balloon moves from the second state toward the first state; and
the first and second flaps are independently movable relative to the shell.

57. The cardiac assist device of claim 56, wherein the first and second flaps extend from the shell proximally.

58. The cardiac assist device of claim 56 or 57, wherein the first and second flaps extend from the shell proximally and in the same circumferential direction relative to the longitudinal axis.

59. The cardiac assist device of any one of claims 39-58, wherein:
the shell includes a membrane carried by the struts;
the membrane defines the aperture; and
the coaptation area includes a portion of the membrane between an inner edge of the struts at the given interstice and an outer edge of the aperture.

60. The cardiac assist device of any one of claims 39-59, wherein an average stiffness of the membrane at the coaptation area is greater than an average stiffness of the flap at the coaptation area.

61. The cardiac assist device of any one of claims 39-60, wherein:
the given interstice is polygonal; and
the aperture is ellipsoid.

62. The cardiac assist device of any one of claims 39-60, wherein the given interstice and the aperture are polygonal.

63. The cardiac assist device of any one of claims 39-60, wherein the given interstice and the aperture are diamond shaped.

64. The cardiac assist device of any one of claims 39-63, wherein:
the aperture is a first aperture;
the cardiac assist device further comprises a second aperture at the given interstice;
the coaptation area is at a perimeter portion of the second aperture;
moving the balloon from the second state toward the first state tends to move fluid into the shell via the second aperture; and
the first and second apertures are spaced apart from one another.

65. The cardiac assist device of any one of claims 39-64, wherein a distance between an inner edge of the struts at the given interstice and an outer edge of the aperture is within a range from 0.5 mm to 4 mm around at least 75% of a full perimeter of the aperture.

66. The cardiac assist device of any one of claims 39-65, wherein a distance between an inner edge of the struts around the given interstice and an outer edge of the aperture varies less than 50% around at least 75% of a full perimeter of the aperture.

67. The cardiac assist device of any one of claims 39-66, wherein:
the flap has an open position in which the flap allows movement of fluid into the shell via the aperture and a closed position in which the flap reduces movement of fluid out of the shell via the aperture; and
the flap is biased toward the closed position.

68. The cardiac assist device of any one of claims 39-67, wherein:
the flap includes:
a hinge portion at which the flap is hingedly connected to the shell,
a contact portion configured to contact the shell at the coaptation area to reduce movement of fluid out of the shell via the aperture while the balloon moves from the first state toward the second state, and
a central portion between the contact portion and the hinge portion; and
an average stiffness of the central portion of the flap is greater than an average stiffness of the contact portion of the flap.

69. The cardiac assist device of claim 68, wherein an average thickness of the central portion of the flap is greater than an average thickness of the contact portion of the flap.

70. The cardiac assist device of claim 68 or 69, wherein the central portion of the flap includes a reinforcing member.

71. The cardiac assist device of claim 70, wherein the central portion of the flap includes reinforcing branches extending from the reinforcing member toward the contact portion of the flap.

72. The cardiac assist device of claim 70 or 71, wherein a composition of the contact portion of the flap is different than a composition of the reinforcing member.

73. The cardiac assist device of claim 72, wherein the contact portion of the flap is at least primarily polymeric, and the reinforcing member is at least primarily metallic.

74. The cardiac assist device of any one of claims 39-73, wherein:
the shell has a concave inner surface at the given interstice;
the flap has a convex outer surface at the given interstice; and
the outer surface of the flap contacts the inner surface of the shell at the coaptation area while the balloon moves from the first state toward the second state.

75. The cardiac assist device of any one of claims 39-74, wherein:
the shell has a curvature at the given interstice; and
the flap has a resting curvature matching the curvature of the shell at the given interstice.

76. The cardiac assist device of any one of claims 39-75, wherein:
the flap includes:
a hinge portion at which the flap is hingedly connected to the shell, and
an end portion opposite to the hinge portion; and
the cardiac assist device further comprises a connection between the shell and the end portion of the flap that restricts movement of the end portion of the flap away from the shell while the balloon moves from the second state toward the first state.

77. The cardiac assist device of claim 76, wherein the connection between the shell and the end portion of the flap is a weld.

78. The cardiac assist device of claim 76, wherein the connection between the shell and the end portion of the flap is a tether or a spring.

79. A method of providing cardiac assist, comprising:
intravascularly advancing a cardiac assist device toward a heart of a patient while the cardiac assist device is in a low-profile delivery state;
locating the cardiac assist device within a cardiovascular lumen of the patient;
moving the cardiac assist device from the delivery state to an expanded treatment state after locating the cardiac assist device within the cardiovascular lumen;
cycling a balloon of the cardiac assist device between a first state and a second state, the balloon being more inflated in the second state than in the first state, wherein the balloon is at least partially disposed within a shell of the cardiac assist device and wherein the shell includes struts arranged in a mesh pattern that defines interstices,
wherein cycling the balloon includes:
moving the balloon from the second state toward the first state such that blood moves into the shell via an aperture of the cardiac assist device, the aperture being at a given one of the interstices, and
moving the balloon from the first state toward the second state such that a flap carried by the shell contacts a coaptation area at a perimeter portion of the aperture thereby reducing movement of blood out of the shell via the aperture.

80. The method of claim 79, wherein cycling the balloon includes cycling the balloon at a rate within a range from 200 to 10,000 complete cycles per minute.

81. The method of claim 78 or 79, wherein:
moving the balloon from the second state toward the first state includes moving the balloon from the second state toward the first state such that blood moves into the shell via first, second, and third apertures of the cardiac assist device, the first, second, and third apertures being at a first one of the interstices, a second one of the interstices, and a third one of the interstices, respectively; and
moving the balloon from the first state toward the second state includes moving the balloon from the first state toward the second state such that the flap contacts first, second, and third coaptation areas at respective perimeter portions of the first, second, and third apertures thereby reducing movement of blood out of the shell via the first, second, and third apertures.

82. The method of any one of claims 79-81, wherein moving the balloon from the second state toward the first state includes moving the balloon from the second state toward the first state while restricting movement of the flap away from the coaptation area via a weld between the shell and an end portion of the flap opposite to a hinge portion of the flap through which the flap is hingedly connected to the shell.

83. The method of any one of claims 79-81, wherein moving the balloon from the second state toward the first state includes moving the balloon from the second state toward the first state while restricting movement of the flap away from the coaptation area via a tether or a spring between the shell and an end portion of the flap opposite to a hinge portion of the flap through which the flap is hingedly connected to the shell.

84. An intravascular blood pump comprising:
a chamber having at least one inlet and an outlet;
a volume displacement member within the chamber configured to move cyclically between a first state and a second state, wherein blood flows into the chamber through the inlet as the volume displacement member transitions from the second state to the first state and blood exits the chamber through the outlet as the volume displacement member transitions from the first state to the second state; and
one or more one-way valves, each one-way valve being associated with at least one inlet, wherein each one-way valve is configured to open and close at a frequency of at least 300 times per minute.

85. The blood pump of claim 84, wherein the one or more one-way valves are configured to allow blood flow into the chamber through the at least one inlet at a rate of at least 30 ml/sec.

86. The blood pump of claim 84, wherein the one or more one-way valves are configured to have a resistance to blood flowing through the at least one inlet of no more than 5×10⁸ kg/(m⁴s).

87. The blood pump of any one of claims 84-86, wherein the volume displacement member comprises a balloon.

88. The blood pump of any one of claims 84-87, wherein at least one of the one-way valves comprises a flap configured to transition between an open state in which the flap is configured to cover the at least one inlet, and a closed state in which the flap is configured to cover the at least one inlet.
